(19) United States
(12) Patent Application Publication
(10) Pub. No.: US 2025/0270605 A1
(43) Pub. Date: Aug. 28, 2025

(54) RECOMBINANT YEAST STRAIN FOR CO-FERMENTATION OF MALTOSE AND GLUCOSE

(71) Applicant: Xylogenics, Inc., Brownsburg, IN (US)

(72) Inventors: Joshua W. Heyen, Brownsburg, IN (US); Nadaraj Palaniappan, Carmel, IN (US); Kathryn A. Houin, Lebanon, IN (US); Ross Cocklin, Keene, NH (US); Mark G. Goebl, Indianapolis, IN (US)

(21) Appl. No.: 19/048,796

(22) Filed: Feb. 7, 2025

Related U.S. Application Data
(63) Continuation-in-part of application No. PCT/US23/71899, filed on Aug. 9, 2023.
(60) Provisional application No. 63/396,466, filed on Aug. 9, 2022.

(51) Int. Cl. C12P 19/02; C12N 9/26; C12N 9/34; C12N 15/81; C12P 19/12

(52) U.S. Cl. CPC C12P 19/02; C12N 9/2425; C12N 9/2428; C12N 15/81; C12P 19/12; C12Y 302/01002; C12Y 302/01003; C12N 2800/102

(57) ABSTRACT

A recombinant yeast strain comprising a strain of S. cerevisiae is disclosed. The strain is capable of co-fermenting maltose and glucose; the strain expresses an exogenous beta-amylase gene. The beta-amylase gene may be fused to a starch binding domain from a gene encoding a glucoamylase. The strain may comprise a glucoamylase gene from P. oxalicum. The strain may comprise a maltogenic alpha-amylase gene from L. plantarum S21. The strain may be used to co-ferment maltose and glucose contained in starch from corn mash with or without added exogenous glucoamylase. The strain has demonstrated enhanced efficiency including accelerated transition from fermentation of glucose to fermentation of maltose. The strain has been shown to facilitate improved/enhanced polysaccharide breakdown. The strain will facilitate efficient co-fermentation of maltose and glucose from feedstocks as to be an alternative to known commercial yeast strains used to produce fuel ethanol.

### CROSS REFERENCE TO RELATED APPLICATIONS

The present application is a continuation-in-part of PCT/International Application No. PCT/US23/71899 filed Aug. 9, 2023, which claims the benefit of priority to U.S. Provisional Application No. 63/396,466, filed Aug. 9, 2022.

The present application claims the benefit of priority to and incorporates by reference in the entirety the disclosure of the following patent applications: (a) PCT/International Application No. PCT/US23/71899 filed Aug. 9, 2023; and (b) U.S. Provisional Application No. 63/396,466, filed Aug. 9, 2022.

### SEQUENCE LISTING

The present application contains a Sequence Listing which has been submitted electronically in XML file format and is hereby incorporated by reference in its entirety. Said XML copy, created on Feb. 7, 2025, is named 982414.000028_XYLO-0004-01-WO.xml is 68 kilobytes in size.

### FIELD

The present invention relates to a recombinant yeast strain for co-fermentation of maltose and glucose.

Aspects of the invention relate to making and using strains of Saccharomyces cerevisiae that are capable of rapidly and efficiently fermenting corn mash into ethanol using an endogenously expressed beta-amylase, a glucoamylase, maltogenic alpha-amylase, and various combinations thereof, all in strains constructed to breakdown large polysaccharides and co-consume maltose and glucose; thereby eliminating the need to convert disaccharides and trisaccharides into glucose through the addition of glucoamylase enzymes to yeast feed stocks.

### BACKGROUND

Various species of Saccharomyces are among the most important industrially grown microorganisms. Long used to leaven bread, produce beer and wine, and as a source of food flavorings and micronutrients, these organisms now play a central role in the production of fuel, facilitating the conversion of sugars to ethanol. A metabolically complex organism, yeast can grow both aerobically and anaerobically as well, if certain nutritional conditions are met. When grown commercially, as in the production of yeast used to support the commercial baking industry, yeasts such as S. cerevisiae are grown in highly aerated fermentation tanks. The growth of yeast under these conditions is manipulated to favor the production of yeast biomass. One way in which this is accomplished is to schedule the addition of sugars, such as D-glucose, and the rate of oxygen transfer to the yeast to encourage aerobic growth. Various strains of Saccharomyces can also be grown under conditions designed to maximize the production of ethanol. Oftentimes, when the object is to maximize the conversion of sugar to ethanol, the level of oxygen in the fermentation vessel is reduced relative to the levels of oxygen used in the vessel during yeast biomass production in order to favor anaerobic growth.

Most strains of Saccharomyces prefer growth on D-glucose although many strains are known to grow on other naturally occurring hexoses and even some disaccharides as well. The ability of different species of Saccharomyces to grow on different sugars and in the presence of different levels of oxygen accounts for much of its commercial utility including the central role that yeast currently plays in the conversion of plant bio-mass into ethanol for various uses including its use as a fuel.

One of the best-known pathways for the production of ethanol by yeast is the fermentation of 6-carbon sugars (hexoses) into ethanol, especially D-glucose. One widely used feedstock for the production of ethanol is the polysaccharide starch. Starch is a simple polymer consisting of chains of D-glucose. Currently, in the United States at least, starch derived from corn kernels is the preferred feed stock for bio-ethanol production by S. cerevisiae.

A single kernel of corn is comprised of ˜65-80 percent starch depending on the growing season and the specific corn variety. Starch in its most basic form is a polymer of many glucose molecules linked through glycosidic bonds. This polymer can take on two basic forms. Amylose is primarily a linear glucose polymer that can contain up to 600 glucose molecules linked together by α-(1,4) linkages. Amylopectin, however, consists of large highly branched glucose polymers that can range in degree of polymerization from hundreds of thousands to millions of glucose units. Glucose units in amylopectin are linked together by both α-(1,4) and α-(1,6) linkages with the latter type providing the branching structure. Together, many amylose and amylopectin molecules intertwine into an ordered superstructure known as a starch granule (looks much like a very small onion with concentric layers). A single kernel of corn contains many starch granules consisting of 70-80 percent amylopectin and 20-30 percent amylose.

Starch granules serve to store chemical energy for the seed in a very compact and recalcitrant state. This allows for a large amount of energy to be packed into a small space while inhibiting the use of this energy reserve by microbes. In this form, starch is unavailable to the cells of the seed for energy and must therefore be broken down by enzymes into metabolizable molecules (monosaccharide and disaccharide sugars, i.e. glucose and maltose). The initial steps in producing fuel ethanol from corn are designed to achieve the same goal; breakdown of corn starch to usable cellular energy. Cellular energy in the form of ATP, NADH and NADPH is produced as yeast ferment starch to ethanol.

The process to extract and hydrolyze corn starch in preparation for yeast fermentation starts when corn is received at the ethanol production facility. Corn is received either directly from the farmer or through other intermediaries at the ethanol plant by rail or truck. Each shipment is tested for quality by monitoring percent moisture, percent foreign particles, and the presence of toxins. Each facility has its own corn standards that must be met to accept a certain corn shipment. Corn of low moisture <=20 percent, low foreign particles, and minimal toxicity enables the most efficient and highest yielding fermentations. However, corn qualities such as percentage starch content, protein content, the amylose to amylopectin ratio, as well as a multitude of other factors drastically affect fermentation yield. These factors vary by region, corn hybrid, weather, farm practices, and other unpredictable variables. It is therefore common to have drastic swings in ethanol plant productivity due to variation in the corn quality from different harvests.

Once corn has been purchased and received, it is either stored on sight or fed directly to a mill. There are two different milling procedures utilized in the United States known as wet milling or dry milling. Over 70 percent of the 13.3 Billion gallons of fuel ethanol made in the United States in 2012 was made using what is called a dry milling or dry grind process. For this reason, the application includes dry milling although the invention disclosed can be used with feed stocks prepared by virtually any milling process.

The milling process includes forming the corn into fine flour using any number of milling technologies. The most common mill utilized is a hammer mill that disrupts and grinds the corn kernel using sharpened shafts (hammers) spinning at high speed around a central axis (think enclosed fan). As the hammers spin, they grind corn entering from the top of the mill until the corn is ground small enough to pass through a screen of a given size. Screen size dictates the particle size of the flour and influences many downstream processes. As flour particle size rises, the downstream enzymatic hydrolysis of the starch becomes less and less complete, ultimately decreasing the amount of sugar available to yeast and the amount of ethanol that can be produced from a given amount of corn. However, creating smaller particle sizes requires more work (energy) as the hammer mill must operate at a higher amperage to break down the particles. Smaller particle sizes also increase soluble solids in thin stillage, reducing centrifuge and evaporator efficiency during co-product feed production (Evaporation is an energy intensive process). For these reasons, milling practices vary across ethanol production facilities; on particles with an average screen, sizes between 2.5 and 3 mm are utilized.

The ground corn flour is then mixed with water at a certain ratio in a slurry mixer. The ratio of water to corn flour determines the solids level of the final fermentation corn mash. The solids level is an important parameter in fuel ethanol production. This ratio ultimately determines the amount of sugar that is supplied to the yeast and therefore determines the maximum ethanol titer that can be achieved when the material is fermented. Today, ethanol producers in the United States typically favor a 32 percent corn flour mixture (32 percent Solids) but solids levels can vary between 28 and 34 percent, depending on facility and season. Fermentations carried out at these solids levels are known as VHG fermentations (for Very High Gravity). The ability to carry out VHG fermentations drastically increases the efficiency of fuel ethanol production but is currently limited to the aforementioned solids levels for several reasons.

In a typical process to produce ethanol from corn, the corn flour and water slurry is mixed with an α-amylase enzyme in a slurry mixer. The enzyme/corn/water mixture (mash) is then pumped to a slurry tank where it is heated to ˜90° C. to gelatinize the starch for hydrolysis by the α-amylase. The α-amylase is an endoenzyme and thus hydrolyzes glycosidic bonds within the starch granule. This action quickly reduces the viscosity of the mash as it de-polymerizes the starch polymer into shorter chain dextrins. Typically, the mash is held in the slurry tank for ˜ 20 minutes and is then sterilized, further gelatinized, and sheared in a jet cooker at 200° C. Jet-cooked mash is then pumped into the liquefaction tanks, treated with a second dose of α-amylase, and held at 80-90° C. for two hours to further break down the starch into dextrins. The mash is then cooled to 30-34° C. and pumped into an 800,000-gallon fermentation tank along with yeast, nutrients, and a second enzyme, glucoamylase, to start a process known as SSF (Simultaneous Saccharification and Fermentation). Glucoamylase is an exo-acting β-amylase that liberates glucose from the non-reducing ends of starch polymers and dextrins. Thus, gluco-amylase ‘spoon feeds’ fermentable sugars to the yeast for fermentation to ethanol. The upstream processing required to produce fermentable sugars from starch for yeast fermentation is time and energy intensive.

Most commonly used glucoamylase enzyme technologies are designed to produce glucose from corn starch at a rate consistent with the rate that yeast will ferment glucose, which is preferred by normal yeast for fermentation. This preference is defined in part by the fact that when presented with a mixture of fermentable sugars, strains of S. cerevisiae are used to produce ethanol ferment glucose first and almost exclusively until virtually all the available glucose is fully consumed. Only after virtually all of glucose is completely consumed will these strains of yeast switch to fermenting other sugars that may be available in the feed stock.

All the glucoamylase enzymes commonly used in the fuel ethanol industry are inhibited to various degrees by the presence of maltose, and maltose is almost always produced to some degree during the breakdown of starch. The accumulation of glucose in the fermenter is also undesirable as it increases the osmolarity of the environment in the fermentation vessel. Most strains of yeast used to produce ethanol are sensitive to the osmolarity of the fermentation environment; high osmolarity can reduce the efficiency of the fermentation and slow or even inhibit the ability of the yeast to produce ethanol. Accordingly, coordinating the rate of glucose production from the breakdown with the rate of glucose consumption by yeast is also necessitated by the need to reduce osmolality of the fermentation environment.

Because the accumulation of high concentrations of glucose in the fermenter broth may lead to stuck fermentations and tremendous yield reductions, traditional fermentation systems limit the rate of starch breakdown to coincide with the rate of yeast glucose fermentation. This limitation reduces the amount of starch that can be broken down and fermented in each 54-hour fermentation and thus limits maximum fermenter yield. Interestingly, maltose, which is also a fermentable sugar that can be produced from corn starch, is half as osmotically stressful to yeast and thus can accumulate to concentrations that are twice the acceptable glucose concentration in a fermenter. Therefore, the rate of starch breakdown can be greatly accelerated by producing the less stressful sugar maltose. Maltose production allows for higher solids to be loaded into a fermenter leading to higher ethanol titers, lower water usage, lower heat usage, and greater margins.

However, maltose fermentation in standard commercial yeast is glucose repressed and thus the efficiency of maltose fermentations is greatly inhibited by the accumulation of even small amounts of glucose in the fermenter using traditional commercial yeast. Thus, glucose repression has prevented the application of high gravity maltose fermentations. Some aspects of the present invention address the apparent difficulties of high gravity maltose fermentations.

### SUMMARY

The present invention relates to a recombinant yeast strain comprising a strain of S. cerevisiae capable of co-fermenting maltose and glucose and an exogenous beta-amylase gene; the strain of S. cerevisiae expresses an exogenous beta-amylase gene. The beta-amylase gene is in the genome of the strain of S. cerevisiae within at least one of (a) intergenic space between YDR439W and YDR440W or (b) the Dubious Open Reading Frame YMR082c or (c) the Dubious Open Reading Frame YCR022C. The beta-amylase gene has at least 80 percent identity to SEQ. ID NO.: 1. The beta-amylase gene may be fused to a DNA sequence encoding a polypeptide comprising an amino acid sequence at least 85 percent identical to SEQ ID. NO.: 9. The exogenous beta-amylase gene may be fused to a starch binding domain from a gene encoding a glucoamylase. The strain may comprise an exogenous glucoamylase gene from P. oxalicum. The glucoamylase gene from P. oxalicum is in the genome of the strain of S. cerevisiae within one of (a) intergenic space between YDR439W and YDR440W or (b) the Dubious Open Reading Frame YMR082c. The glucoamylase gene from P. oxalicum comprises a sequence having at least 80 percent identity to SEQ. ID NO.: 3. The strain may comprise an exogenous maltogenic alpha-amylase gene from L. plantarum S21. The exogenous maltogenic alpha-amylase gene from L. plantarum S21 is inserted into the genome of the strain of S. cerevisiae within the Dubious Open Reading Frame YCR022C. The maltogenic alpha-amylase gene from L. plantarum S21 may comprise a sequence having at least 80 percent identity to SEQ. ID NO.: 5. The strain may be used in a medium comprising starch with a DP4+ component with a weight of approximately 21.5 percent of total volume in corn mash with approximately 33.53 percent weight-to-weight solids without added exogenous glucoamylase; the DP4+ component may comprise a weight of no more than 10.7 percent of total volume after 12 hours of fermentation. The strain may be used in a medium comprising starch with a DP4+ component with a weight of approximately 21.1 percent of total volume in corn mash with approximately 33.62 percent weight-to-weight solids without added exogenous glucoamylase; the DP4+ component may comprise a weight of no more than 9.4 percent of total volume after 12 hours of fermentation; the medium comprises ethanol with a weight of no less than approximately 13.4 percent of total volume after 24 hours of fermentation. The strain may be used in a medium comprising starch with a DP4+ component with a weight of approximately 21.1 percent of total volume in corn mash with approximately 33.62 percent weight-to-weight solids with exogenous glucoamylase at a ratio of 0.015 percent weight-to-weight solids; the medium comprises ethanol with a weight of no less than approximately 11.1 percent of total volume after 24 hours of fermentation; the medium comprises glucose with a weight of no more than approximately 0.3 percent of total volume after 24 hours of fermentation. Co-fermenting maltose and glucose comprises simultaneous fermentation of maltose and glucose. Co-fermenting maltose and glucose comprises accelerated transition from fermentation of glucose to fermentation of maltose.

The present invention relates to a vector comprising an exogenous beta-amylase gene with a sequence having at least 80 percent homology and/or identity to SEQ. ID NO.: 1.

The present invention relates to a recombinant yeast strain comprising a strain of S. cerevisiae capable of simultaneously fermenting maltose and glucose and an exogenous beta-amylase gene; the strain of S. cerevisiae expresses an exogenous beta-amylase gene. The exogenous beta-amylase gene may be overexpressed. The exogenous beta-amylase gene may be present in one or more copies per cell. The beta-amylase gene may be integrated into the genome of the strain of S. cerevisiae. The beta-amylase gene may be inserted into the genome of the strain of S. cerevisiae within one or all or a combination of the following regions: intergenic space between YDR439W and YDR440W, the Dubious Open Reading Frame YMR082c or the Dubious Open Reading Frame YCR022C. The exogenous beta-amylase gene may be from P. flexia. and/or has at least 80 percent homology to SEQ. ID NO.: 1. The exogenous beta-amylase gene may be fused to a starch binding domain from a glucoamylase gene of Asperigillus niger encoding a polypeptide comprising an amino acid sequence at least 85 percent identical to SEQ ID. NO.: 9. The ability to simultaneously ferment maltose and glucose may be conferred by a constitutively active, glucose-insensitive MALX3 allele encoding a polypeptide comprising an amino acid sequence at least 85 percent identical to SEQ ID. NO.: 10. The recombinant yeast strain may comprise an exogenous glucoamylase gene from P. oxalicum. The exogenous glucoamylase gene from P. oxalicum gene may be overexpressed. The exogenous glucoamylase gene from P. oxalicum may be present in one or more copies per cell. The glucoamylase gene from P. oxalicum may be inserted into the genome of the strain of S. cerevisiae within one or both of the following regions: intergenic space between YDR439W and YDR440W, the Dubious Open Reading Frame YMR082c. The exogenous glucoamylase gene from P. oxalicum comprises a sequence having at least 80 percent homology to SEQ. ID NO.: 3 and the exogenous beta-amylase gene from P. flexa comprises a sequence having at least 80 percent homology to SEQ. ID NO.: 1. The exogenous glucoamylase gene from P. oxalicum comprises a sequence having at least 85 percent homology to SEQ. ID NO.: 3 and the exogenous beta-amylase gene from P. flexa comprises a sequence having at least 85 percent homology to SEQ. ID NO.: 1. The exogenous glucoamylase gene from P. oxalicum comprises a sequence having at least 90 percent homology to SEQ. ID NO.: 3 and the beta-amylase gene from P. flexa comprises a sequence having at least 90 percent homology to SEQ. ID NO.: 1. The exogenous glucoamylase gene from P. oxalicum comprises a sequence having at least 95 percent homology to SEQ. ID NO.: 3 and the exogenous beta-amylase gene from P. flexa comprises a sequence having at least 95 percent homology to SEQ. ID NO.: 1. The exogenous glucoamylase gene from P. oxalicum comprises a sequence having SEQ. ID NO.: 3 and the exogenous beta-amylase gene from P. flexa comprises a sequence having SEQ. ID NO.: 1. The recombinant yeast strain may comprise an exogenous maltogenic alpha-amylase gene from L. plantarum S21. The exogenous maltogenic alpha-amylase gene from L. plantarum S21 gene may be overexpressed. The exogenous maltogenic alpha-amylase gene from L. plantarum S21 may be inserted into the genome of the strain of S. cerevisiae within the Dubious Open Reading Frame YCR022C. The exogenous maltogenic alpha-amylase gene from L. plantarum S21 may be inserted into the genome of the strain of S. cerevisiae within a region encoding the Dubious Open Reading Frame YCR022C. The maltogenic alpha-amylase gene from L. plantarum S21 comprises a sequence having at least 80 percent homology to SEQ. ID NO.: 5 and the exogenous glucoamylase gene from P. oxalicum comprises a sequence having at least 80 percent homology to SEQ. ID NO.: 3 and the exogenous beta-amylase gene from P. flexa comprises a sequence having at least 80 percent homology to SEQ. ID NO.: 1. The exogenous maltogenic alpha-amylase gene from L. plantarum S21 comprises a sequence having at least 85 percent homology to SEQ. ID NO.: 5 and the exogenous glucoamylase gene from P. oxalicum comprises a sequence having at least 85 percent homology to SEQ. ID NO.: 3 and the exogenous beta-amylase gene from P. flexa comprises a sequence having at least 85 percent homology to SEQ. ID NO.: 1. The exogenous maltogenic alpha-amylase gene from L. plantarum S21 comprises a sequence having at least 90 percent homology to SEQ. ID NO.: 5 and the exogenous glucoamylase gene from P. oxalicum comprises a sequence having at least 90 percent homology to SEQ. ID NO.: 3 and the exogenous beta-amylase gene from P. flexa comprises a sequence having at least 90 percent homology to SEQ. ID NO.: 1. The exogenous maltogenic alpha-amylase gene from L. plantarum S21 comprises a sequence having at least 95 percent homology to SEQ. ID NO.: 5 and the exogenous glucoamylase gene from P. oxalicum comprises a sequence having at least 95 percent homology to SEQ. ID NO.: 3 and the exogenous beta-amylase gene from P. flexa comprises a sequence having at least 95 percent homology to SEQ. ID NO.: 1. The exogenous maltogenic alpha-amylase gene from L. plantarum S21 comprises a sequence having SEQ. ID NO.: 5 and the exogenous glucoamylase gene from P. oxalicum comprises a sequence having SEQ. ID NO.: 3 and the exogenous beta-amylase gene from P. flexa comprises a sequence having SEQ. ID NO.: 1. The strain of S. cerevisiae may be haploid, diploid, or has a ploidy number greater than two. The recombinant yeast strain may be created using genetic engineering; the recombinant yeast strain may be genetically modified. The recombinant yeast strain may be capable of fermenting maltose as well as disaccharides and trisaccharides comprised of glucose while simultaneously improving the efficiency and speed of glucose fermentation and eliminating the requirement for supplemental glucoamylase.

The present invention relates to a vector comprising an exogenous beta-amylase gene from P. flexa that comprises a sequence having at least 80 percent, at least 85 percent, at least 90 percent, at least 95 percent, at least 98 percent, or 100 percent percent homology or identity to SEQ. ID NO.: 1. The vector may comprise an exogenous glucoamylase gene from P. oxalicum that comprises a sequence having at least 80 percent, at least 85 percent, at least 90 percent, at least 95 percent, at least 98 percent, or 100 percent percent homology or identity to SEQ. ID NO.: 3. The vector may comprise an exogenous maltogenic alpha-amylase gene from L. plantarum S21 that comprises a sequence having at least 80 percent, at least 85 percent, at least 90 percent, at least 95 percent, at least 98 percent, or 100 percent homology or identity to SEQ. ID NO.: 5. The exogenous maltogenic alpha amylase gene from L. plantarum S21 and/or the beta-amylase gene from P. flexa and/or the glucoamylase gene from P. oxalicum are maintained and expressed in a haploid, diploid, or polyploid of the strain of S. cerevisiae. The vector may be expressed in the strain of S. cerevisiae as a single copy or multiple copies.

The present invention relates a method of producing a recombinant yeast strain comprising the step of integrating an exogenous beta-amylase from P. flexa having at least 80 percent homology to SEQ. ID NO.: 1 and/or an exogenous glucoamylase gene from P. oxalicum having at least 80 percent homology to SEQ. ID NO.: 3 and/or an exogenous maltogenic alpha-amylase gene from L. plantarum S21 having at least 80 percent homology to SEQ. ID NO.: 5 into the genome of a strain of S. cerevisiae.

Various strains of S. cerevisiae are the industry standard strain for commercial production of fuel ethanol from grains such as corn. One widely used strain of S. cerevisiae is the commercially available ETHANOL RED™ strain of yeast. This strain has a robust system for utilizing glucose and includes a functional MAL2 locus which enables the strain to ferment maltose. Aspects of the present invention consists of modified ETHANOL RED™ strains of yeast in which the rate of polysaccharide hydrolysis has increased, maltose and DP3 sugar fermentation have been modestly improved and glucose fermentation rates have increased, thereby improving fermentation of high maltose syrups and maltose/glucose mixtures and furthermore eliminating the requirement for exogenous glucoamylase enzyme. One such example that improves maltose and glucose co-consumption through modification of the maltose uptake system is further discussed and described in U.S. patent application Ser. No. 17/261,454, filed on Jan. 19, 2021. One iteration of the example was strain F15. In one embodiment of the present invention, the F15 strain was modified to also contain an exogenous beta-amylase along with an exogenous glucoamylase. All amylase genes were codon optimized for best expression in S. cerevisiae. The beta-amylase from Priestia flexa is carried on the same cassette as the Penicillium oxalicum glucoamylase and integrated in two unique sites of the S. cerevisiae genome, Neutral Landing Sites 3 and 9. Neutral Landing Site 3 is located on Chromosome XIII and overlaps dubious Open Reading Frame YMR082C while Neutral Landing Site 9 is located on Chromosome IV within the intergenic space between YDR439W and YDR440W. This strain has been given the name F23.

In another embodiment, the F15 strain was modified to also contain an exogenous beta-amylase, an exogenous glucoamylase and an exogenous maltogenic alpha-amylase. All amylase genes were codon optimized for best expression in S. cerevisiae. In addition to the insertion of the beta-amylase from P. flexa and glucoamylase from P. oxalicum being integrated into Neutral Landing Sites 3 and 9, a maltogenic alpha-amylase from Lactobacillus plantarum S21 is encoded on a cassette along with another copy of beta-amylase from P. flexa and integrated into Neutral Landing Site 7. Neutral Landing Site 7 is located on Chromosome III and overlaps dubious Open Reading Frame YCR022C. This strain has been given the name F22.

In another embodiment, the beta amylase is not identical to SEQ. ID NO.: 1, but its encoded protein products share 95 percent similarity with the protein products encoded in SEQ. ID NO.: 1 and shown as SEQ. ID NO.: 2. Still other embodiments include integration of a beta-amylase from P. flexa (SEQ. ID NO.: 1) and glucoamylase from P. oxalicum (SEQ. ID NO.: 2) into other yeast strains important for ethanol production. In another embodiment, the beta-amylase, the glucoamylase and the maltogenic alpha amylase are not integrated into the yeast genome; instead, they are expressed and maintained on a plasmid. The plasmid may either be maintained at one copy per cell or as multiple copies per cell. This is dictated by the plasmid type. The plasmid may contain a CEN/ARS sequence allowing replication and faithful transmission to daughter cells. Furthermore, the beta-amylase, the glucoamylase and the alpha amylase may be expressed from the same plasmid or two or three separate plasmids.

A first embodiment includes a recombinant yeast strain, comprising a strain of S. cerevisiae, a functional MAL1 gene cluster, a constitutive and glucose insensitive MALX3 gene, an exogenous beta-amylase from P. flexa; wthe strain of S. cerevisiae expresses the MAL1 gene cluster, the MALX3 gene, and the exogenous beta-amylase gene from P. flexa.

A second embodiment includes the recombinant yeast strain according to the first embodiment, wthe beta-amylase from P. flexa is overexpressed.

A third embodiment includes the recombinant yeast strain according to any one of the first and second embodiments, wthe beta-amylase from P. flexa is present in more than one copy per cell.

A fourth embodiment includes the recombinant yeast strain according to any one of the first to third embodiments, wthe beta amylase from P. flexa is integrated into the genome of the strain of S. cerevisiae.

A fifth embodiment includes the recombinant yeast strain according to any one of the first to fourth embodiments, wthe beta amylase from P. flexa is fused with the starch binding domain of the A. niger glucoamylase gene.

A sixth embodiment includes the recombinant yeast strain according to any one of the first to fifth embodiments, wthe beta amylase from P. flexa is inserted into the genome of the strain of S. cerevisiae within a region encoding the Dubious Open Reading Frame YMR082C.

A seventh embodiment includes the recombinant yeast strain according to any one of the first to fifth embodiments, wthe beta amylase from P. flexa is inserted into the genome of the strain of S. cerevisiae within an intergenic region between Open Reading Frames YDR439W and YDR440W.

An eighth embodiment includes the recombinant yeast strain according to any one of the first to fifth embodiments, wthe beta amylase from P. flexa is inserted into the genome of the strain of S. cerevisiae within a region encoding Dubious Open Reading Frame YCR022C.

A ninth embodiment includes the recombinant yeast strain according to the fourth or fifth embodiments, wthe beta amylase from P. flexa is integrated into the genome at different positions on more than one chromosome.

A tenth embodiment includes the recombinant yeast strain according to the ninth embodiment, wthe beta amylase from P. flexa is inserted into two places of the genome of the strain of S. cerevisiae, a first region encoding the Dubious Open Reading Frame YMR082C and a second intergenic region between Open Reading Frames YDR439W and YDR440W.

An eleventh embodiment includes the recombinant yeast strain according to the ninth embodiment, wthe beta amylase from P. flexa is inserted into three places of the genome of the strain of S. cerevisiae, a first region encoding the Dubious Open Reading Frame YMR082C, a second intergenic region between Open Reading Frames YDR439W and YDR440W, and a third encoding Dubious Open Reading Frame YCR022C.

A twelfth embodiment includes the recombinant yeast strain according to any one of the first to eleventh embodiments, further comprising an exogenous glucoamylase gene from P. oxalicum.

A thirteenth embodiment includes the recombinant yeast strain according to the twelfth embodiment, wthe exogenous glucoamylase gene from P. oxalicum is overexpressed.

A fourteenth embodiment includes the recombinant yeast strain according to any one of the twelfth or thirteenth embodiments, wthe exogenous glucoamylase gene from P. oxalicum is present in more than one copy per cell.

A fifteenth embodiment includes the recombinant yeast strain according to any one of the twelfth to fourteenth embodiments, wthe glucoamylase from P. oxalicum is integrated into the genome of the strain of S. cerevisiae.

An sixteenth embodiment includes the recombinant yeast strain according to the fifteenth embodiment, wthe exogenous glucoamylase gene from P. oxalicum is inserted into the genome of the strain of S. cerevisiae within a region encoding the Dubious Open Reading Frame YMR082C.

A seventeenth embodiment includes the recombinant yeast strain according to the fifteenth embodiment, wthe exogenous glucoamylase gene from P. oxalicum is inserted into the genome of the strain of S. cerevisiae within an intergenic region between Open Reading Frames YDR439W and YDR440W.

An eighteenth embodiment includes the recombinant yeast strain according to the fifteenth embodiment, wthe exogenous glucoamylase gene from P. oxalicum is integrated into the genome at different positions on more than one chromosome.

A nineteenth embodiment includes the recombinant yeast strain according to the eighteenth embodiment, wthe exogenous glucoamylase gene from P. oxalicum is inserted into two places of the genome of the strain of S. cerevisiae, a first region encoding the Dubious Open Reading Frame YMR082C and a second intergenic region between Open Reading Frames YDR439W and YDR440W.

A twentieth embodiment includes the recombinant yeast strain according to any one of the first to nineteenth embodiments, further comprising an exogenous maltogenic alpha-amylase gene from L. plantarum S21.

A twenty-first embodiment includes the recombinant yeast strain according to the twentieth embodiment, wthe exogenous maltogenic alpha-amylase gene from L. plantarum S21 is overexpressed.

A twenty-second embodiment includes the recombinant yeast strain according to any one of the twentieth and twenty-first embodiments, wthe exogenous maltogenic alpha-amylase gene from L. plantarum S21 is present in more than one copy per cell.

A twenty-third embodiment includes the recombinant yeast strain according to any one of the twentieth to twenty-second embodiments, wthe maltogenic alpha-amylase gene from L. plantarum S21 is integrated into the genome of the strain of S. cerevisiae.

A twenty-fourth embodiment includes the recombinant yeast strain according to any one of the twentieth to twenty-third embodiments, wthe maltogenic alpha-amylase gene from L. plantarum S21 is inserted into the genome of the strain of S. cerevisiae within a region encoding Dubious Open Reading Frame YCR022C.

A twenty-fifth embodiment includes the recombinant yeast strain according to any one of the twentieth to twenty-fourth embodiments, wthe exogenous beta amylase gene from P. flexa comprises a sequence having at least 80 percent homology to SEQ. ID NO.: 2 and the exogenous glucoamylase gene from P. oxalicum comprises a sequence having at least 80 percent homology to SEQ. ID NO.: 4 and the exogenous maltogenic alpha amylase gene from L. plantarum S21 comprises a sequence having at least 80 percent homology to SEQ. ID NO.: 6. The exogenous beta amylase gene from P. flexa may comprise a sequence having at least 81 percent, at least 82 percent, at least 83 percent, and/or at least 84 percent homology to SEQ. ID NO.: 2 and the exogenous glucoamylase from P. oxalicum may comprise a sequence having at least 81 percent, at least 82 percent, at least 83 percent, and/or at least 84 percent homology to SEQ. ID NO.: 4 and the exogenous maltogenic alpha amylase gene from L. plantarum S21 comprises a sequence having at least 81 percent, at least 82 percent, at least 83 percent, and/or at least 84 percent homology to SEQ. ID NO.: 6.

A twenty-sixth embodiment includes the recombinant yeast strain according to any one of the twentieth to twenty-fifth embodiments, wthe exogenous beta amylase gene from P. flexa comprises a sequence having at least 85 percent homology to SEQ. ID NO.: 2 and the exogenous glucoamylase gene from P. oxalicum comprises a sequence having at least 85 percent homology to SEQ. ID NO.: 4 and the exogenous maltogenic alpha amylase gene from L. plantarum S21 comprises a sequence having at least 85 percent homology to SEQ. ID NO.: 6. The exogenous beta amylase gene from P. flexa may comprise a sequence having at least 86 percent, at least 87 percent, at least 88 percent, and/or at least 89 percent homology to SEQ. ID NO.: 2 and the exogenous glucoamylase from P. oxalicum may comprise a sequence having at least 86 percent, at least 87 percent, at least 88 percent, and/or at least 89 percent homology to SEQ. ID NO.: 4 and the exogenous maltogenic alpha amylase gene from L. plantarum S21 comprises a sequence having at least 86 percent, at least 87 percent, at least 88 percent, and/or at least 89 percent homology to SEQ. ID NO.: 6.

A twenty-seventh embodiment includes the recombinant yeast strain according to any one of the twentieth to twenty-seventh embodiments, wthe exogenous beta amylase gene from P. flexa comprises a sequence having at least 90 percent homology to SEQ. ID NO.: 2 and the exogenous glucoamylase gene from P. oxalicum comprises a sequence having at least 90 percent homology to SEQ. ID NO.: 4 and the exogenous maltogenic alpha amylase gene from L. plantarum S21 comprises a sequence having at least 90 percent homology to SEQ. ID NO.: 6. The exogenous beta amylase gene from P. flexa may comprise a sequence having at least 91 percent, at least 92 percent, at least 93 percent, and/or at least 94 percent homology to SEQ. ID NO.: 2 and the exogenous glucoamylase from P. oxalicum may comprise a sequence having at least 91 percent, at least 92 percent, at least 93 percent, and/or at least 94 percent homology to SEQ. ID NO.: 4 and the exogenous maltogenic alpha amylase gene from L. plantarum S21 comprises a sequence having at least 91 percent, at least 92 percent, at least 93 percent, and/or at least 94 percent homology to SEQ. ID NO.: 6.

A twenty-eighth embodiment includes the recombinant yeast strain according to any one of the twentieth to twenty-seventh embodiments, wthe exogenous beta amylase gene from P. flexa comprises a sequence having at least 95 percent homology to SEQ. ID NO.: 2 and the exogenous glucoamylase gene from P. oxalicum comprises a sequence having at least 95 percent homology to SEQ. ID NO.: 4 and the exogenous maltogenic alpha amylase gene from L. plantarum S21 comprises a sequence having at least 95 percent homology to SEQ. ID NO.: 6. The exogenous beta amylase gene from P. flexa may comprise a sequence having at least 96 percent, at least 97 percent, at least 98 percent, and/or at least 99 percent homology to SEQ. ID NO.: 2 and the exogenous glucoamylase from P. oxalicum may comprise a sequence having at least 96 percent, at least 97 percent, at least 98 percent, and/or at least 99 percent homology to SEQ. ID NO.: 4 and the exogenous maltogenic alpha amylase gene from L. plantarum S21 comprises a sequence having at least 96 percent, at least 97 percent, at least 98 percent, and/or at least 99 percent homology to SEQ. ID NO.: 6.

A twenty-nineth embodiment includes the recombinant yeast strain according to any one of the twentieth to twenty-eighth embodiments, wthe exogenous beta amylase gene from P. flexa comprises a sequence having SEQ. ID NO.: 1 and the exogenous glucoamylase gene from P. oxalicum comprises a sequence having SEQ. ID NO.: 3 and the exogenous maltogenic alpha amylase gene from L. plantarum S21 comprises a sequence having SEQ. ID NO.: 5.

A thirtieth embodiment includes the recombinant yeast strain according to any one of the first to the twenty-nineth embodiments, wthe strain of S. cerevisiae is haploid, diploid, or has a ploidy number greater than two.

A thirty-first embodiment includes the recombinant yeast strain according to any one of the first to the thirtieth embodiments, wthe recombinant yeast strain is made using genetic engineering or wthe recombinant yeast strain is genetically modified.

A thirty-second embodiment includes any one of the first to the thirty-first embodiments, wthe recombinant yeast strain is capable of fermenting maltose as well as disaccharides and trisaccharides comprised of glucose while simultaneously improving the efficiency and speed of glucose fermentation and eliminating the requirement for supplemental glucoamylase.

A thirty-thrid embodiment includes a vector comprising a beta amylase gene from P. flexa that comprises a sequence having at least 80 percent, at least 85 percent, at least 90 percent, at least 95 percent, at least 98 percent, or at least 100 percent homology or identity to SEQ. ID NO.: 1. The beta amylase gene from P. flexa may comprise a sequence having at least 81 percent, at least 82 percent, at least 83 percent, at least 84 percent, at least 86 percent, at least 87 percent, at least 88 percent, at least 89 percent, at least 91 percent, at least 92 percent, at least 93 percent, at least 94 percent, at least 96 percent, at least 97 percent, at least 98 percent, and/or at least 99 percent homology to SEQ. ID NO.: 1.

A thirty-fourth embodiment includes the vector according to the thirty-third embodiment, further comprising a glucoamylase gene from P. oxalicum that comprises a sequence having at least 80 percent, at least 85 percent, at least 90 percent, at least 95 percent, at least 98 percent, or at least 100 percent homology or identity to SEQ. ID NO.: 3. The glucoamylase gene from P. oxalicum may comprise a sequence having at least 81 percent, at least 82 percent, at least 83 percent, at least 84 percent, at least 86 percent, at least 87 percent, at least 88 percent, at least 89 percent, at least 91 percent, at least 92 percent, at least 93 percent, at least 94 percent, at least 96 percent, at least 97 percent, at least 98 percent, and/or at least 99 percent homology to SEQ. ID NO.: 3.

A thirty-fifth embodiment includes the vector according to the thirty-third embodiment, further comprising a maltogenic alpha-amylase gene from L. plantarum S21 that comprises a sequence having at least 80 percent, at least 85 percent, at least 90 percent, at least 95 percent, at least 98 percent, or at least 100 percent homology or identity to SEQ. ID NO.: 5. The alpha-amylase gene from L. plantarum S21 may comprise a sequence having at least 81 percent, at least 82 percent, at least 83 percent, at least 84 percent, at least 86 percent, at least 87 percent, at least 88 percent, at least 89 percent, at least 91 percent, at least 92 percent, at least 93 percent, at least 94 percent, at least 96 percent, at least 97 percent, at least 98 percent, and/or at least 99 percent homology to SEQ. ID NO.: 5.

A thirty-sixth embodiment includes the vector according to any one of the thirty-third to the thirty-fifth embodiments, wthe beta amylase gene from P. flexa and/or the maltogenic alpha amylase gene from L. plantarum S21 and/or the glucoamylase gene the glucoamylase gene from P. oxalicum are maintained and expressed in a haploid, diploid, or polyploid of a strain of S. cerevisiae.

A thirty-seventh embodiment includes the vector according to any one of the thirty-third to the thirty-sixth embodiments, wthe vector is expressed in the strain of S. cerevisiae as a single copy or multiple copies. Consistent with these embodiments, the vector and/or plasmid may either be maintained at one copy per cell or as multiple copies per cell.

### FIGURES

FIG. 1 is a schematic diagram of yeast strain construction according to an exemplary embodiment.

FIG. 2 is a schematic diagram of a plasmid for yeast strain construction according to an exemplary embodiment.

FIG. 3 is a schematic diagram of attributes for yeast strain construction according to an exemplary embodiment.

FIG. 4 is a schematic diagram of attributes for yeast strain construction according to an exemplary embodiment.

FIG. 5 is a schematic diagram of a plasmid for yeast strain construction according to an exemplary embodiment.

FIG. 6 is a schematic diagram of attributes for yeast strain construction according to an exemplary embodiment.

FIG. 7 is a schematic diagram of efficacy of yeast strains according to an exemplary embodiment.

FIGS. 8A through 8E are schematic diagrams of efficacy of yeast strains according to an exemplary embodiment.

### DESCRIPTION

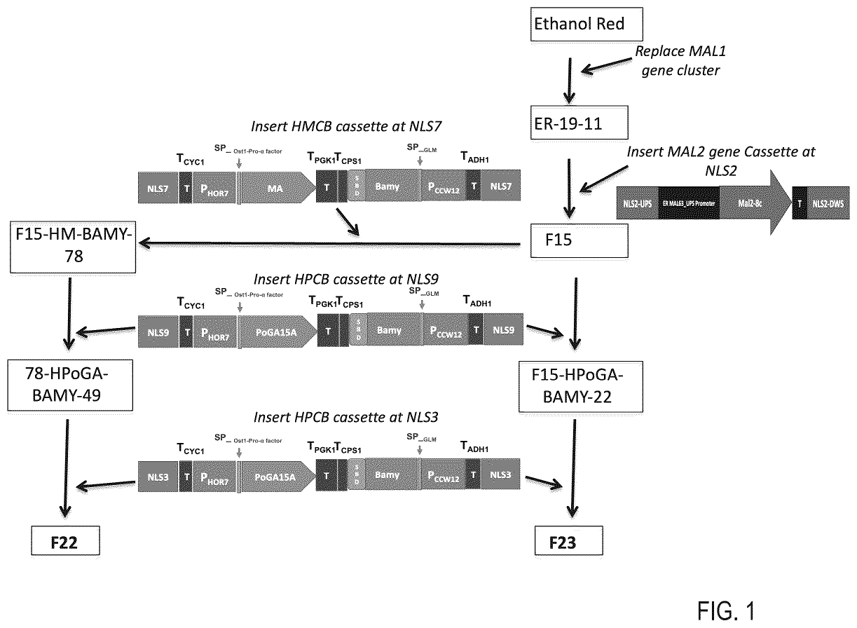

As indicated schematically in FIGS. 1, 7 and 8A-8E, a recombinant yeast strain comprising a strain of S. cerevisiae has been shown to be capable of co-fermenting maltose and glucose and an exogenous beta-amylase gene; the strain of S. cerevisiae expresses an exogenous beta-amylase gene. See generally FIGS. 1 through 12 and TABLE A.

The construction of F23 strain was achieved by two consecutive integrations of selected beta-amylase and maltogenic alpha-amylase enzymes cassettes at neutral landing sites (NLS) of 9 and 3, respectively, in the parent strain, F15, as previously described in U.S. Pat. No. 12,203,111.

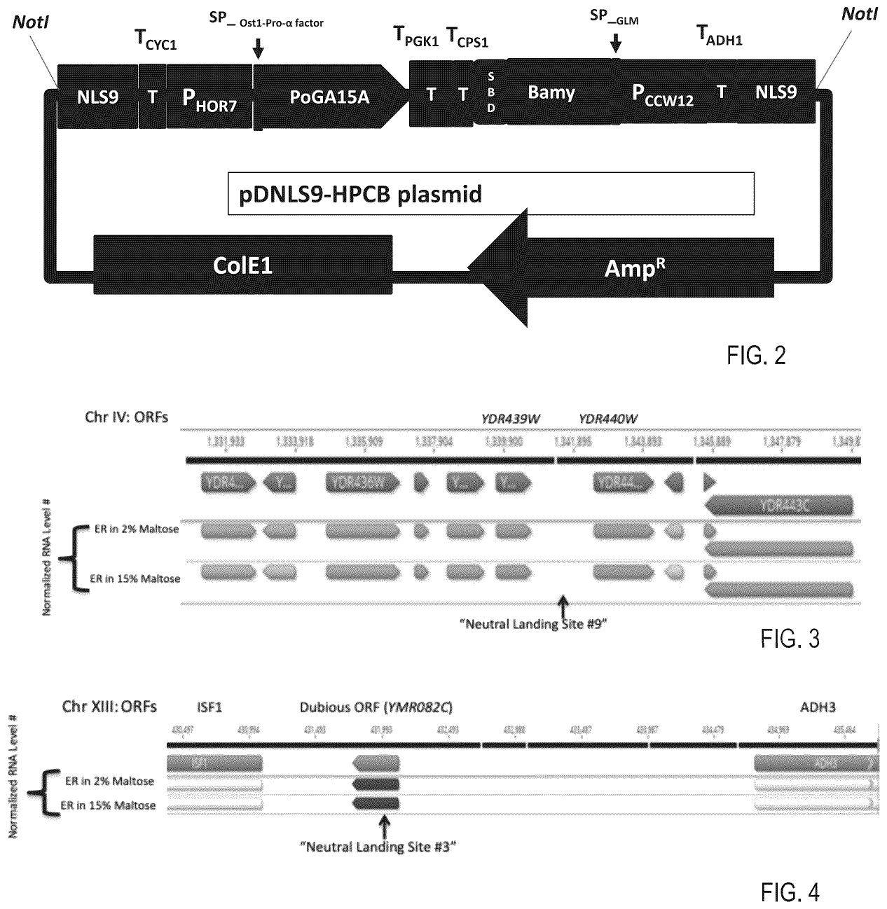

The first integration cassette, termed NLS9-HPCB, includes one glucoamylase, PoGA of P. oxalicum, under the HOR7 promoter and a beta-amylase from P. flexa whose expression is controlled by the CCW12 promoter. The PoGA gene was fused with a short Ost1-Pro a factor signal sequence which directs its passage through the secretory system and ultimate extracellular secretion. The BAMY gene was fused with GLM signal peptide sequence to direct its passage through the secretory system and ultimate extracellular secretion. A starch binding domain was also appended to the C-terminus of BAMY. Both amylase gene sequences used in the construction of HPCB cassette along with signal peptide sequences and starch binding domain sequence were codon optimized for S. cerevisiae and synthesized as gblock DNA fragments (IDT, Coralville, IA, USA). The CCW12 and HOR7 promoters, along with CYC1, PGK1 and ADH1 terminator sequences were PCR amplified from the genomic DNA of ETHANOL RED™ strain of yeast using Q5 PCR reaction mixture (New England Biolabs). The overlapping PCR fragments were gel purified and then cloned into Pmel linearized target vector backbone of pDNLS9-HPCB (FIG. 2 and SEQ ID NO.: 7) using a HiFi DNA assembly kit as recommended in the manufacturer's protocol (New England Biolabs). The correct vector assembly with desired genetic components was verified by PCR and sequencing. The DNA of verified HPCB plasmid was digested with Not1 restriction enzyme and gel purified as linear DNA fragments for integration into the designated Neutral Landing Site 9 of selected S. cerevisiae strains using MAD7/CRISPR technology (Liu et al. 2019) and technical documents at www.inscripta.com). Briefly, the linear DNA fragment of HPCB cassette and plasmid DNA expressing both the MAD7 nuclease and NLS9-targeting gRNA were transformed into S. cerevisiae according to a previously published protocol (Gietz and Woods 2006). We confirmed integration and MAD7 plasmid loss through PCR. A starch hydrolysis assay is also used to confirm integration of the amylases as this improves raw starch degradation. This resulted in the creation of intermediate strain F15-HPoGA-BAMY-22. Neutral Landing Site 9 (NLS9) was selected as the primary site of HPCB cassette integration for several regions. First, to avoid disrupting any important genetic elements, a locale on chromosome IV between ORFS YDR439W and YDR440W was chosen. Genome-wide RNA expressions were measured in Fermentis ETHANOL RED™ strains of yeast fermenting either maltose or glucose at both high (15 percent) and low (2 percent) concentrations. Under all conditions tested the genes neighboring NLS9 are expressed at moderate levels indicating that this is a region amenable to Pol II transcription under a wide variety of conditions (FIG. 3).

The second intermediate strain 15-HPoGA-BAMY-22 was transformed with a similar HPCB cassette, termed NLS3-HPCB, that targets integration into Neutral Landing Site #3 (NLS3). Briefly, the linear DNA fragment of NLS3-HPCB cassette and plasmid DNA expressing both the MAD7 nuclease and NLS3-targeting gRNA were transformed into S. cerevisiae according to a previously published protocol (Gietz and Woods 2006). Transformation and clone selection were carried out as described above and the final strain was verified by PCR and starch hydrolysis assay. This resulted in the creation of subject microorganism strain F-23. Neutral Landing Site 3 (NLS3) was selected as the second site of HPCB cassette integration for several regions. First, to avoid disrupting any important genetic elements, a locale on chromosome XIII overlapping dubious ORF YMR082C was chosen. YMR082C is unlikely to encode a functional protein. Genome-wide RNA expressions were measured in ETHANOL RED™ strain of yeast (FERMENTIS) fermenting either maltose or glucose at both high (15 percent) and low (2 percent) concentrations. Under all conditions tested the genes neighboring NLS3 are expressed at moderate levels indicating that this is a region amenable to Pol II transcription under a wide variety of conditions (FIG. 4).

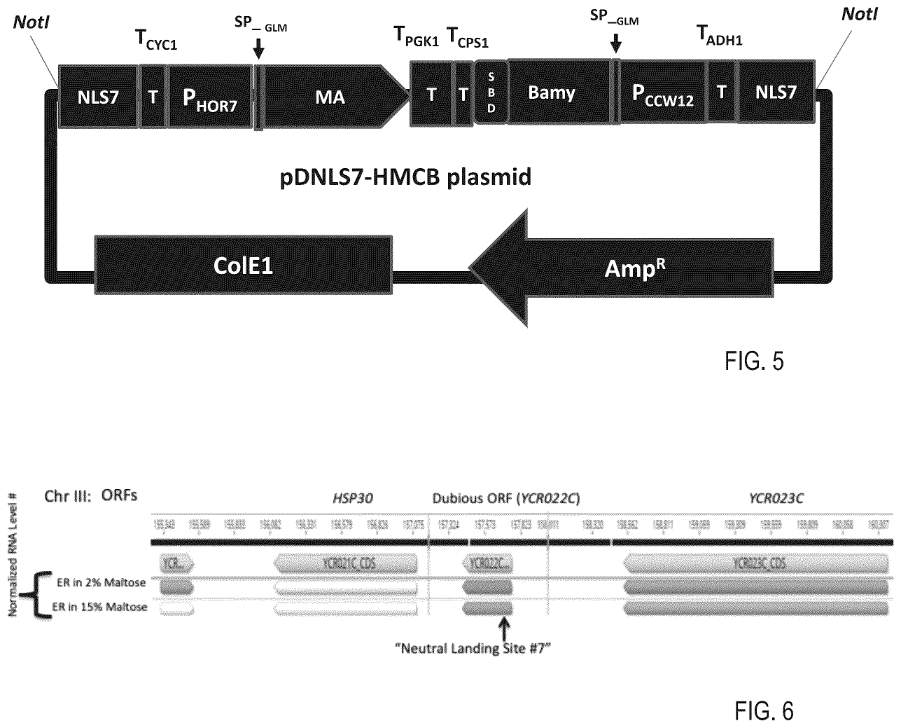

As shown in FIG. 1, F15 was first transformed with the HMCB cassette to create strain F15-HM-BAMY-78 which is an intermediate strain for F-22 strain construction. The initial step of F-22 strain construction was achieved by integration of selected maltogenic alpha-amylase and beta-amylase enzymes at neutral landing site 7. The integration cassette, termed HMCB, includes one maltogenic alpha-amylase, MA of L. plantarum, under the HOR7 promoter and a beta-amylase from P. flexa whose expression is controlled by the CCW12 promoter. Both amylase gene sequences used in the construction of HMCB cassette along with signal peptide sequences and a starch binding domain sequence were codon optimized for S. cerevisiae and synthesized as gblock DNA fragments (IDT, Coralville, IA, USA). The CCW12 and HOR7 promoters, along with CYC1, PGK1 CPS1 and ADH1 terminator sequences were PCR amplified from the genomic DNA of ETHANOL RED™ strain of yeast using Q5 PCR reaction mixture (New England Biolabs). The overlapping PCR fragments were gel purified and then cloned into Pmel linearized target vector backbone of pDNLS7-HMCB (FIG. 5 and SEQ. ID NO.: 8) using a HiFi DNA assembly kit as recommended in the manufacturer's protocol (New England Biolabs). The correct vector assembly with desired genetic components was verified by PCR and sequencing.

The DNA of verified HMCB gene cassette was digested with Not1 restriction enzyme and gel purified as linear DNA fragments for integration into the designated Neutral Landing Site 7 using MAD7/CRISPR technology. As described above, the linear DNA fragment of HPCB cassette and plasmid DNA expressing both the MAD7 nuclease and NLS7-targeting gRNA were transformed into S. cerevisiae according to a previously published protocol (Gietz and Woods 2006). Transformation and clone selection were carried out as described above and the final strain was verified by PCR and starch hydrolysis assay. This resulted in the creation intermediate strain F15-HM-BAMY-78.

Neutral Landing Site (NLS7) was selected as the site of HMHG cassette integration for several regions. First, to avoid disrupting any important genetic elements, a site on chromosome III overlapping the dubious open reading frame YCR022C but sufficiently distant from other annotated genes was chosen. Genome-wide RNA expressions were measured in ETHANOL RED™ strain of yeast (FERMENTIS) fermenting either maltose or glucose at both high (15 percent) and low (2 percent) concentrations. Under all conditions tested the genes neighboring NLS7 are expressed at moderate levels indicating that this is a region amenable to Pol II transcription under a wide variety of conditions (FIG. 6).

The construction of the F-22 yeast strain from F15-HM-BAMY-78 strain parallels the steps of F-23 construction from intermediate strain F15 (FIG. 1). Briefly, one copy of the HPCB cassette was integrated into NLS9 of strain F15-HM-BAMY-78 to create strain 78-HPoGA-BAMY-49. A second copy of the HPCB cassette was integrated into NLS3 to create subject microorganism strain F-22. All plasmid construction and yeast strain transformation were carried out as described above.

### EXAMPLES

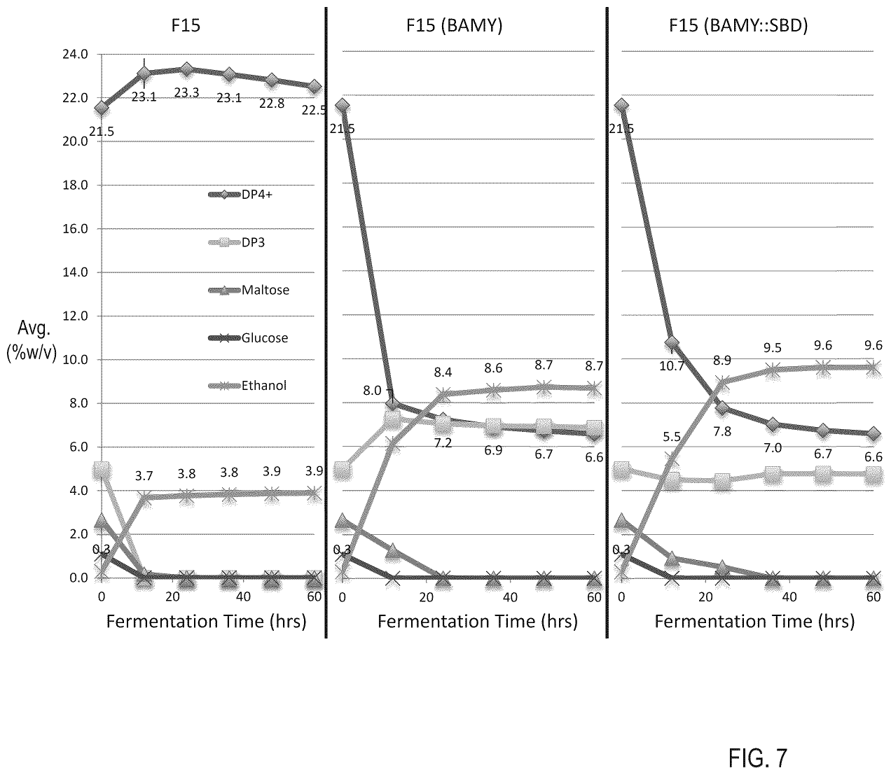

To demonstrate the advantages of the P. flexia beta-amylase and A. niger starch binding domain fusion enzyme (BAMY-SBD) we compared three strains: F15 (Control), F15 expressing P. flexia beta-amylase (F15-BAMY) and F15 expressing the fusion enzyme (F15-BAMY:: SBD). The strains were inoculated at time zero into liquid corn mash slurry containing 33.53 percent solids and no supplemental glucoamylase. The F15 strain expresses no exogenous amylases and is unable to breakdown the polysaccharides (FIG. 7). It consumes the small amounts of maltose and glucose present at fermentation start to produce 3.89 percent ethanol. The F15-BAMY and F15-BAMY:: SBD strains are able to breakdown much of the DP4+sugars, producing various DP3 sugars, maltose and isomaltose which are ultimately fermented into ethanol (FIG. 7). The BAMY:: SBD construct is superior to the BAMY alone in its ability to produce more fermentable DP3 products, allowing the strain to end fermentation with lower DP3s and ˜1 percent higher final ethanol (FIG. 7).

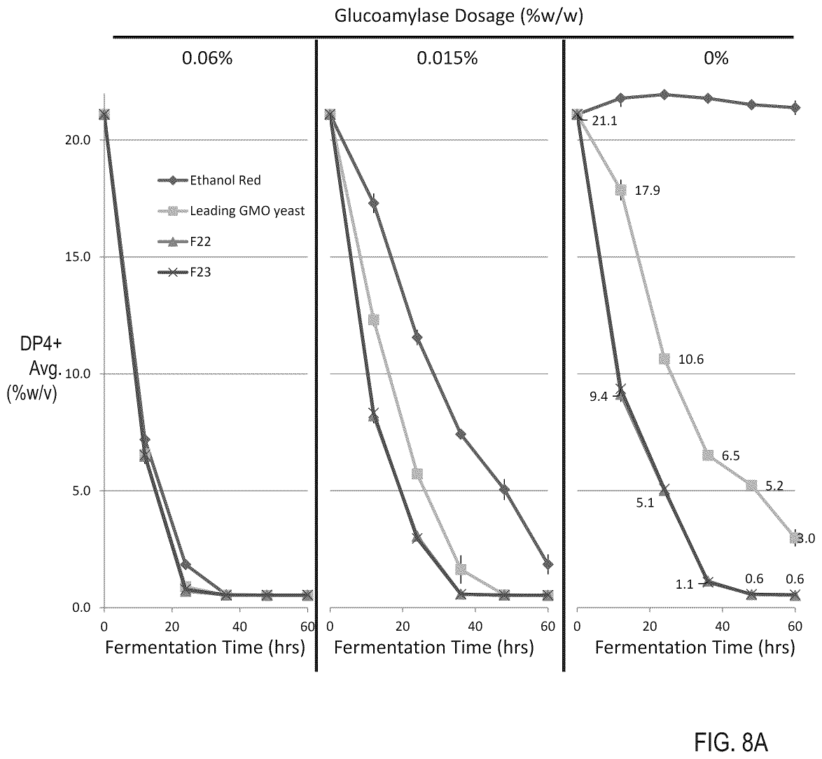

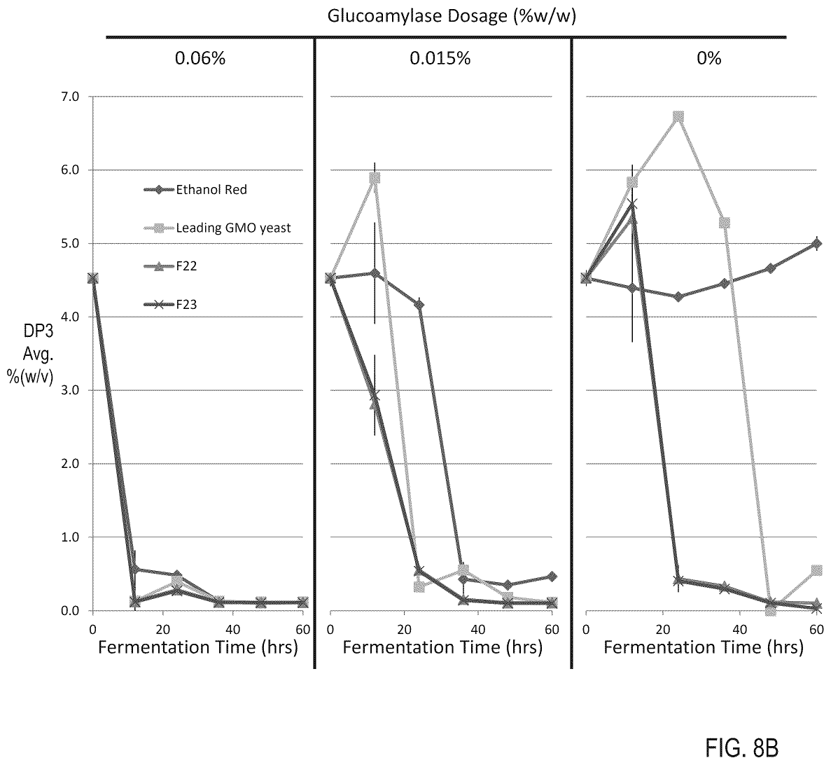

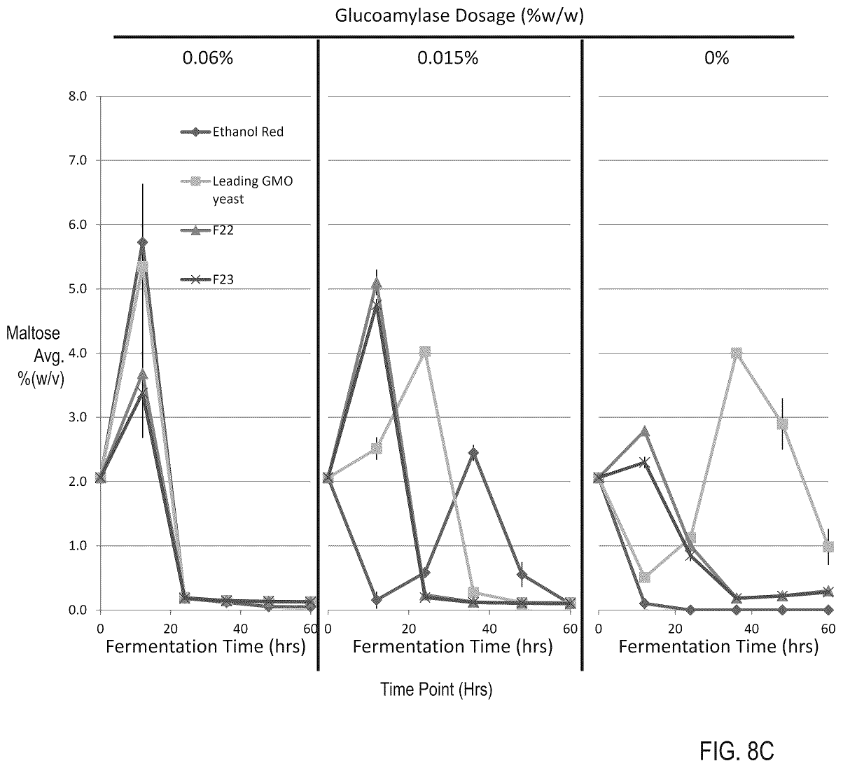

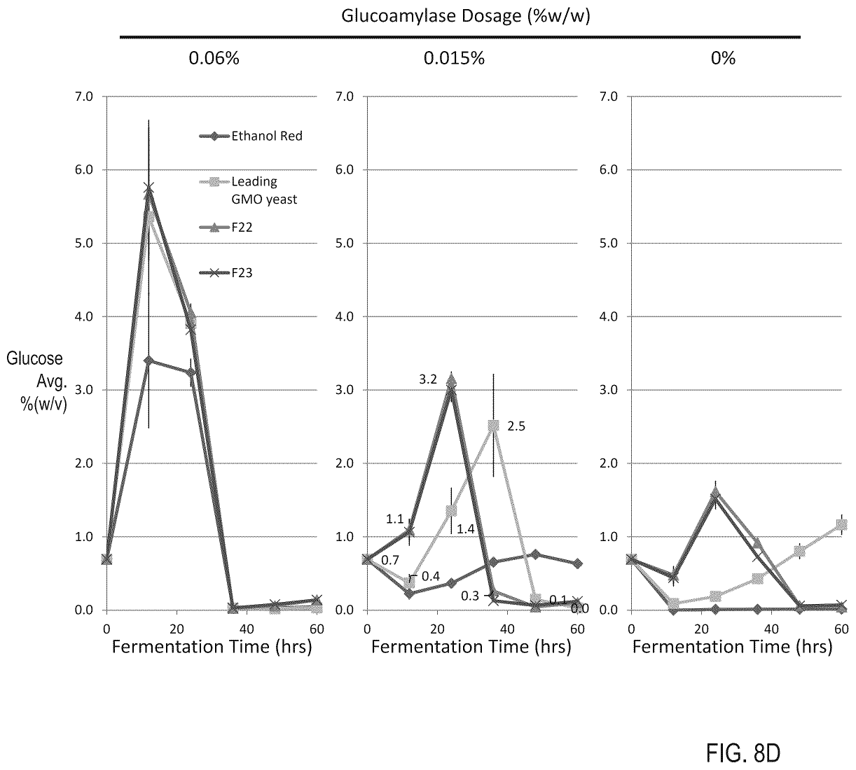

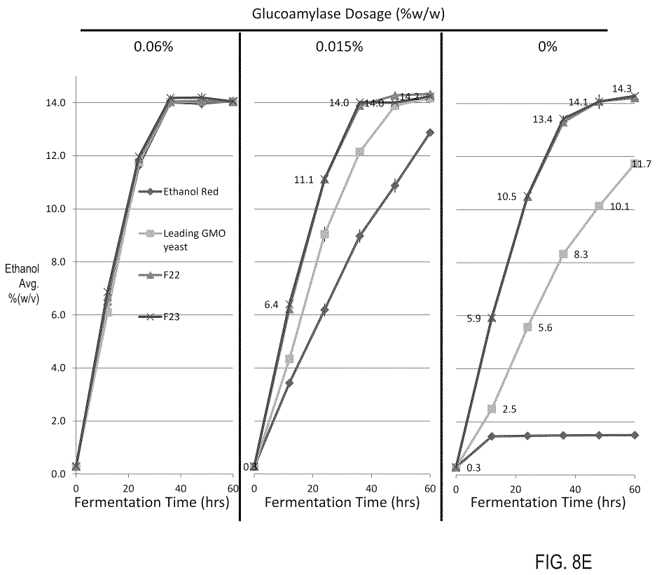

Strains F-22, F-23, ETHANOL RED™ strain of yeast and an industry GMO yeast strain, were inoculated at time zero into liquid corn mash slurry containing 33.62 percent solids. DP4+, DP3, maltose, glucose and ethanol concentrations were measured at various time points over the course of 60 hours. To demonstrate the capacity for fermentations at reduced levels of exogenous glucoamylase (GA), the fermentations were carried out at three different concentrations of exogenous glucoamylase Ultra F (INNOVA™ family of enzymes/glucoamylase from Novozymes of Franklinton, NC), specifically 0, 0.015 percent, and 0.06 percent (weight/weight). Fermentations at 0.06 percent GA serve as the benchmark as that is considered to be full dose GA. All strains supplied full dose GA are able to break down all fermentable DP4+polysaccharides, although F22, F23 and the leading GMO yeast process these sugars more quickly than ETHANOL RED™ strain of yeast (FIG. 8A). F22, F23 and leading GMO yeast consume all DP4+polysaccharides by 24 hours while ETHANOL RED™ strain of yeast requires 36 hours to complete DP4+breakdown (FIG. 8A). At quarter GA dose (0.015 percent) DP4+breakdown for ETHANOL RED™ strain of yeast and leading GMO yeast becomes significantly slower than the F22 and F23 strains (FIG. 8A). F22, F23 and leading GMO yeast do breakdown all DP4+at quarter GA dose while 1.85 percent w/v DP4+remain after 60 hours of fermentation with ETHANOL RED™ strain of yeast (FIG. 8A). As expected, ETHANOL RED™ strain of yeast is unable to breakdown DP4+ in the absence of GA (FIG. 8A). F22 and F23 do not require GA to break down all DP4+while the leading GMO yeast leaves 2.99 percent DP4+after 60 hours (FIG. 8A). The rate of DP4+breakdown is significantly faster with F22 or F23 strains when compared to the leading GMO yeast in both 0.015 percent and 0 GA fermentations (FIG. 8A).

All strains consume DP3 sugars very quickly in fermentations with 0.06 percent GA, presumably as the glucoamylase works to convert DP3 to glucose and various DP2 sugars. At the 0.015 percent GA, the DP3 levels remain much lower in F22 and F23 strains as compared to ETHANOL RED™ strain of yeast and the leading GMO yeast (FIG. 8B). We believe this is due to the synergistic relationship among beta-amylase:: SBD fusion, P. oxalicum glucoamylase and the maltophilic properties of these strains. Furthermore, in runs without GA, DP3 levels peak earlier and finish much lower in the F22 and F23 fermentations compared with the leading GMO yeast (FIG. 8B).

At full dose GA, maltose accumulates through the 12 hour time point in all strains and then consumption outpaces production through the remainder of the ferm. Maltose levels remain below 0.2 percent w/v at the 24 hour time point until completion (FIG. 8C). F22 and F23 maltose levels reach their maximum values of 3.68 percent and 3.38 percent respectively, at 12 hours post-ferm start. This is significantly less than ETHANOL RED™ strain of yeast and the leading GMO yeast due to the enhanced maltose uptake system of F22 and F23 (FIG. 8C). At 0.015 percent GA, maltose peaks again at 12 hours in F22 and F23 while it peaks at 24 hours in leading GMO yeast and 36 hours in ETHANOL RED™ strain of yeast (FIG. 8C). It should be noted that the maximum maltose concentrations are greater than 0.6 percent w/v higher in F22 and F23 fermentations compared with the leading GMO yeast. The maltophilic properties of F22 and F23 allow more maltose production and consumption at reduced GA in F22 and F23. Again, F22 and F23 performance is superior in fermentations with no GA as the maltose accumulates until 12 hours but is depleted by 24 hours and maintained at very low levels throughout the remainder of the fermentation (FIG. 8C). Maltose accumulates very late in the run in leading GMO yeast at zero GA and this strain leaves ˜1 percent residual maltose at the end of the run as cells of this strain die or transition to stationary phase growth (FIG. 8C).

The activities of exogenous amylases expressed by F22, F23 and leading GMO yeast breakdown polysaccharides quicker than ETHANOL RED™ strain of yeast at full dose GA; thereby producing more glucose earlier in the fermentation (FIG. 8D). These strains are particularly adept at quickly consuming high levels of glucose. They all bring the level of glucose down to near baseline by 36 hours and from that point forward its consumption keeps pace with or outpaces production (FIG. 8D). Furthermore, F22 and F23 are able to breakdown starch into glucose and consume nearly all of the glucose during fermentations with 0.015 percent GA (FIG. 8D). Maximum glucose concentration is achieved after 24 hours for fermentations with 0.015 percent GA using F22 or F23 yeast while the maximum concentration is reached at 36 hours with the leading GMO yeast (FIG. 8D). Earlier accumulation of glucose allows F22 and F23 additional time to ferment glucose to ethanol and is manifested as a higher overall ethanol yield at 0.015 percent GA (FIGS. 7D-E). A similar phenomenon can be observed for the fermentations with no GA. Maximum glucose concentration occurs after 24 hours for F22 and F23 strains which is taken down to a baseline level by 48 hours (FIG. 8D). In contrast, glucose doesn't accumulate in the leading GMO yeast until the end of fermentation, presumably as cells of the leading GMO yeast die or transition to stationary phase (FIG. 8D). Without the supplemental GA, the amylases expressed by the leading GMO yeast strain are insufficient to provide sufficient glucose for robust growth and fermentation.

Ethanol production is nearly identical for all strains when supplemented with full dose GA. However, at a GA dose of 0.015 percent ETHANOL RED™ strain of yeast is unable to breakdown all fermentable sugars and therefore produces less total ethanol (FIG. 7E). The rate of ethanol production is much faster with F22 and F23 as they reach 13.9 percent and 14.0 percent ethanol by 36 hours (FIG. 8E). Comparably, the leading GMO yeast only reach 12.15 percent ethanol by 36 hours but does finish fermentation with a comparable level of ethanol to F22 and F23 (FIG. 8E). F22 and F23 do not require GA to finish a fermentation within 60 hours (FIG. 8E). Most notably, F22 and F23 strains are able to finish fermentation at nearly identical concentrations of ethanol at full dose, quarter dose and no GA (FIG. 8E). This is due to the activities of inserted P. flexa beta-amylase, P. oxalicum glucoamylase and, in the case of F22, the L. bacillus maltogenic alpha-amylase. Taken together, F22 and F23 are able to ferment liquid corn mash slurries of 33.62 percent solids to completion with any exogenous glucoamylase. F22 and F23 can ferment the same amount of corn starch and produce nearly identical levels of ethanol at the end of fermentation either with or without GA supplementation.

### Exemplary Embodiments-A

According to an exemplary embodiment shown schematically in the FIGURES, construction and efficacy of a recombinant yeast strain comprising a strain of S. cerevisiae capable of fermenting maltose and glucose; the strain may include an exogenous beta-amylase gene. FIG. 1 schematically illustrates the strains, cassettes and neutral landing sites utilized during construction of the F22 and F23 strains. FIG. 2 schematically illustrates the features of plasmid pDNLS9-HPCB. FIG. 3 schematically illustrates details of the genomic features and gene expression profiles around dubious ORFs YDR439W and YDR440W termed “Neutral Landing Site #9”, the site of HPCB. YDR439W and YDR440W are dubious Open reading frames whose transcripts do not code for functional proteins. FIG. 4 schematically illustrates details of the genomic features and gene expression profiles around dubious ORF YMR082C, termed “Neutral Landing Site #3”, the site of HGHP. YMR082C is a dubious Open reading frame whose transcript does not code for a functional protein. FIG. 5 schematically illustrates the features of plasmid pDNLS7-HMCB. FIG. 6 schematically illustrates details of the genomic features and gene expression profiles around dubious ORF YCR022C, termed “Neutral Landing Site #7”, the site of HMHG. YMR022C is a dubious Open reading frame whose transcript does not code for a functional protein. FIG. 7 schematically illustrates the changes in DP4+, DP3, maltose, glucose and ethanol levels from F15, F15-BAMY and F15-BAMY:: SBD yeast strains under fermentation conditions with corn mash containing 33.53 percent solids and no supplemental glucoamylase. FIG. 8A schematically illustrates the changes in DP4+ levels from ETHANOL RED™ strain of yeast, F22, F23 and leading GMO yeast strains under standard fermentation conditions when corn mash is treated with either a none or 0.015 percent, or 0.06 percent solution of Ultra F (INNOVA™ family of enzymes/glucoamylase from Novozymes of Franklinton, NC). FIG. 8B schematically illustrates the changes in DP3 levels from Ethanol Red yeast, F22, F23 and leading GMO yeast strains under standard fermentation conditions when corn mash is treated with either a none or 0.015 percent, or 0.06 percent solution of Ultra F (INNOVA™ family of enzymes/glucoamylase from Novozymes of Franklinton, NC). FIG. 8C schematically illustrates the changes in maltose levels from Ethanol Red™ strain of yeast, F22, F23 and leading GMO yeast strains under standard fermentation conditions when corn mash is treated with either a none or 0.015 percent, or 0.06 percent solution of Ultra F (INNOVA™ family of enzymes/glucoamylase from Novozymes of Franklinton, NC). FIG. 8D schematically illustrates the changes in glucose levels from Ethanol Red yeast, F22, F23 and leading GMO yeast strains under standard fermentation conditions when corn mash is treated with either a none or 0.015 percent, or 0.06 percent solution of Ultra F (INNOVA™ family of enzymes/glucoamylase from Novozymes of Franklinton, NC). FIG. 8E schematically illustrates the changes in ethanol levels from Ethanol Red yeast, F22, F23 and leading GMO yeast strains under standard fermentation conditions when corn mash is treated with either a none or 0.015 percent, or 0.06 percent solution of Ultra F (INNOVA™ family of enzymes/glucoamylase from Novozymes of Franklinton, NC).

According to an exemplary embodiment a recombinant yeast strain may comprise a strain of S. cerevisiae capable of simultaneously fermenting maltose and glucose and an exogenous beta-amylase gene; the strain of S. cerevisiae expresses an exogenous beta-amylase gene. The exogenous beta-amylase gene may be overexpressed. The exogenous beta-amylase gene may be present in one or more copies per cell. The beta-amylase gene may be integrated into the genome of the strain of S. cerevisiae. The beta-amylase gene may be inserted into the genome of the strain of S. cerevisiae within one or all or a combination of the following regions: intergenic space between YDR439W and YDR440W, the Dubious Open Reading Frame YMR082c or the Dubious Open Reading Frame YCR022C. The exogenous beta-amylase gene may be from P. flexia. and/or has at least 80 percent homology to SEQ. ID NO.: 1. The exogenous beta-amylase gene may be fused to a starch binding domain from a glucoamylase gene of Asperigillus niger encoding a polypeptide comprising an amino acid sequence at least 85 percent identical to SEQ ID. NO.: 9. The ability to simultaneously ferment maltose and glucose may be conferred by a constitutively active, glucose-insensitive MALX3 allele encoding a polypeptide comprising an amino acid sequence at least 85 percent identical to SEQ ID. NO.: 10. The recombinant yeast strain may comprise an exogenous glucoamylase gene from P. oxalicum. The exogenous glucoamylase gene from P. oxalicum gene may be overexpressed. The exogenous glucoamylase gene from P. oxalicum may be present in one or more copies per cell. The glucoamylase gene from P. oxalicum may be inserted into the genome of the strain of S. cerevisiae within one or both of the following regions: intergenic space between YDR439W and YDR440W, the Dubious Open Reading Frame YMR082c. The exogenous glucoamylase gene from P. oxalicum comprises a sequence having at least 80 percent homology to SEQ. ID NO.: 3 and the exogenous beta-amylase gene from P. flexa comprises a sequence having at least 80 percent homology to SEQ. ID NO.: 1. The exogenous glucoamylase gene from P. oxalicum comprises a sequence having at least 85 percent homology to SEQ. ID NO.: 3 and the exogenous beta-amylase gene from P. flexa comprises a sequence having at least 85 percent homology to SEQ. ID NO.: 1. The exogenous glucoamylase gene from P. oxalicum comprises a sequence having at least 90 percent homology to SEQ. ID NO.: 3 and the beta-amylase gene from P. flexa comprises a sequence having at least 90 percent homology to SEQ. ID NO.: 1. The exogenous glucoamylase gene from P. oxalicum comprises a sequence having at least 95 percent homology to SEQ. ID NO.: 3 and the exogenous beta-amylase gene from P. flexa comprises a sequence having at least 95 percent homology to SEQ. ID NO.: 1. The exogenous glucoamylase gene from P. oxalicum comprises a sequence having SEQ. ID NO.: 3 and the exogenous beta-amylase gene from P. flexa comprises a sequence having SEQ. ID NO.: 1. The recombinant yeast strain may comprise an exogenous maltogenic alpha-amylase gene from L. plantarum S21. The exogenous maltogenic alpha-amylase gene from L. plantarum S21 gene may be overexpressed. The exogenous maltogenic alpha-amylase gene from L. plantarum S21 may be inserted into the genome of the strain of S. cerevisiae within the Dubious Open Reading Frame YCR022C. The exogenous maltogenic alpha-amylase gene from L. plantarum S21 may be inserted into the genome of the strain of S. cerevisiae within a region encoding the Dubious Open Reading Frame YCR022C. The maltogenic alpha-amylase gene from L. plantarum S21 comprises a sequence having at least 80 percent homology to SEQ. ID NO.: 5 and the exogenous glucoamylase gene from P. oxalicum comprises a sequence having at least 80 percent homology to SEQ. ID NO.: 3 and the exogenous beta-amylase gene from P. flexa comprises a sequence having at least 80 percent homology to SEQ. ID NO.: 1. The exogenous maltogenic alpha-amylase gene from L. plantarum S21 comprises a sequence having at least 85 percent homology to SEQ. ID NO.: 5 and the exogenous glucoamylase gene from P. oxalicum comprises a sequence having at least 85 percent homology to SEQ. ID NO.: 3 and the exogenous beta-amylase gene from P. flexa comprises a sequence having at least 85 percent homology to SEQ. ID NO.: 1. The exogenous maltogenic alpha-amylase gene from L. plantarum S21 comprises a sequence having at least 90 percent homology to SEQ. ID NO.: 5 and the exogenous glucoamylase gene from P. oxalicum comprises a sequence having at least 90 percent homology to SEQ. ID NO.: 3 and the exogenous beta-amylase gene from P. flexa comprises a sequence having at least 90 percent homology to SEQ. ID NO.: 1. The exogenous maltogenic alpha-amylase gene from L. plantarum S21 comprises a sequence having at least 95 percent homology to SEQ. ID NO.: 5 and the exogenous glucoamylase gene from P. oxalicum comprises a sequence having at least 95 percent homology to SEQ. ID NO.: 3 and the exogenous beta-amylase gene from P. flexa comprises a sequence having at least 95 percent homology to SEQ. ID NO.: 1. The exogenous maltogenic alpha-amylase gene from L. plantarum S21 comprises a sequence having SEQ. ID NO.: 5 and the exogenous glucoamylase gene from P. oxalicum comprises a sequence having SEQ. ID NO.: 3 and the exogenous beta-amylase gene from P. flexa comprises a sequence having SEQ. ID NO.: 1. The strain of S. cerevisiae may be haploid, diploid, or has a ploidy number greater than two. The recombinant yeast strain may be created using genetic engineering or the recombinant yeast strain may be genetically modified. The recombinant yeast strain may be capable of fermenting maltose as well as disaccharides and trisaccharides comprised of glucose while simultaneously improving the efficiency and speed of glucose fermentation and eliminating the requirement for supplemental glucoamylase.

According to an exemplary embodiment a recombinant yeast strain may comprise a strain of S. cerevisiae capable of simultaneously fermenting maltose and glucose and an exogenous beta-amylase gene; the strain of S. cerevisiae expresses an exogenous beta-amylase gene. The exogenous beta-amylase gene may be overexpressed. The exogenous beta-amylase gene may be present in one or more copies per cell. The beta-amylase gene may be integrated into the genome of the strain of S. cerevisiae. The beta-amylase gene may be inserted into the genome of the strain of S. cerevisiae within one or all or a combination of the following regions: intergenic space between YDR439W and YDR440W, the Dubious Open Reading Frame YMR082c or the Dubious Open Reading Frame YCR022C. The exogenous beta-amylase gene may be from P. flexia. and/or has at least 80 percent identity to SEQ. ID NO.: 1. The exogenous beta-amylase gene may be fused to a starch binding domain from a glucoamylase gene of Asperigillus niger encoding a polypeptide comprising an amino acid sequence at least 85 percent identical to SEQ ID. NO.: 9. The ability to simultaneously ferment maltose and glucose may be conferred by a constitutively active, glucose-insensitive MALX3 allele encoding a polypeptide comprising an amino acid sequence at least 85 percent identical to SEQ ID. NO.: 10. The recombinant yeast strain may comprise an exogenous glucoamylase gene from P. oxalicum. The exogenous glucoamylase gene from P. oxalicum gene may be overexpressed. The exogenous glucoamylase gene from P. oxalicum may be present in one or more copies per cell. The glucoamylase gene from P. oxalicum may be inserted into the genome of the strain of S. cerevisiae within one or both of the following regions: intergenic space between YDR439W and YDR440W, the Dubious Open Reading Frame YMR082c. The exogenous glucoamylase gene from P. oxalicum comprises a sequence having at least 80 percent identity to SEQ. ID NO.: 3 and the exogenous beta-amylase gene from P. flexa comprises a sequence having at least 80 percent identity to SEQ. ID NO.: 1. The exogenous glucoamylase gene from P. oxalicum comprises a sequence having at least 85 percent identity to SEQ. ID NO.: 3 and the exogenous beta-amylase gene from P. flexa comprises a sequence having at least 85 percent identity to SEQ. ID NO.: 1. The exogenous glucoamylase gene from P. oxalicum comprises a sequence having at least 90 percent identity to SEQ. ID NO.: 3 and the beta-amylase gene from P. flexa comprises a sequence having at least 90 percent identity to SEQ. ID NO.: 1. The exogenous glucoamylase gene from P. oxalicum comprises a sequence having at least 95 percent identity to SEQ. ID NO.: 3 and the exogenous beta-amylase gene from P. flexa comprises a sequence having at least 95 percent identity to SEQ. ID NO.: 1. The exogenous glucoamylase gene from P. oxalicum comprises a sequence having SEQ. ID NO.: 3 and the exogenous beta-amylase gene from P. flexa comprises a sequence having SEQ. ID NO.: 1. The recombinant yeast strain may comprise an exogenous maltogenic alpha-amylase gene from L. plantarum S21. The exogenous maltogenic alpha-amylase gene from L. plantarum S21 gene may be overexpressed. The exogenous maltogenic alpha-amylase gene from L. plantarum S21 may be inserted into the genome of the strain of S. cerevisiae within the Dubious Open Reading Frame YCR022C. The exogenous maltogenic alpha-amylase gene from L. plantarum S21 may be inserted into the genome of the strain of S. cerevisiae within a region encoding the Dubious Open Reading Frame YCR022C. The maltogenic alpha-amylase gene from L. plantarum S21 comprises a sequence having at least 80 percent identity to SEQ. ID NO.: 5 and the exogenous glucoamylase gene from P. oxalicum comprises a sequence having at least 80 percent identity to SEQ. ID NO.: 3 and the exogenous beta-amylase gene from P. flexa comprises a sequence having at least 80 percent identity to SEQ. ID NO.: 1. The exogenous maltogenic alpha-amylase gene from L. plantarum S21 comprises a sequence having at least 85 percent identity to SEQ. ID NO.: 5 and the exogenous glucoamylase gene from P. oxalicum comprises a sequence having at least 85 percent identity to SEQ. ID NO.: 3 and the exogenous beta-amylase gene from P. flexa comprises a sequence having at least 85 percent identity to SEQ. ID NO.: 1. The exogenous maltogenic alpha-amylase gene from L. plantarum S21 comprises a sequence having at least 90 percent identity to SEQ. ID NO.: 5 and the exogenous glucoamylase gene from P. oxalicum comprises a sequence having at least 90 percent identity to SEQ. ID NO.: 3 and the exogenous beta-amylase gene from P. flexa comprises a sequence having at least 90 percent identity to SEQ. ID NO.: 1. The exogenous maltogenic alpha-amylase gene from L. plantarum S21 comprises a sequence having at least 95 percent identity to SEQ. ID NO.: 5 and the exogenous glucoamylase gene from P. oxalicum comprises a sequence having at least 95 percent identity to SEQ. ID NO.: 3 and the exogenous beta-amylase gene from P. flexa comprises a sequence having at least 95 percent identity to SEQ. ID NO.: 1. The exogenous maltogenic alpha-amylase gene from L. plantarum S21 comprises a sequence having SEQ. ID NO.: 5 and the exogenous glucoamylase gene from P. oxalicum comprises a sequence having SEQ. ID NO.: 3 and the exogenous beta-amylase gene from P. flexa comprises a sequence having SEQ. ID NO.: 1. The strain of S. cerevisiae may be haploid, diploid, or has a ploidy number greater than two. The recombinant yeast strain may be created using genetic engineering or the recombinant yeast strain may be genetically modified. The recombinant yeast strain may be capable of fermenting maltose as well as disaccharides and trisaccharides comprised of glucose while simultaneously improving the efficiency and speed of glucose fermentation and eliminating the requirement for supplemental glucoamylase.

According to an exemplary embodiment, a vector may comprise an exogenous beta-amylase gene from P. flexa that comprises a sequence having at least 80 percent, at least 85 percent, at least 90 percent, at least 95 percent, at least 98 percent, or 100 percent percent homology or identity to SEQ. ID NO.: 1. The vector may comprise an exogenous glucoamylase gene from P. oxalicum that comprises a sequence having at least 80 percent, at least 85 percent, at least 90 percent, at least 95 percent, at least 98 percent, or 100 percent percent homology or identity to SEQ. ID NO.: 3. The vector may comprise an exogenous maltogenic alpha-amylase gene from L. plantarum S21 that comprises a sequence having at least 80 percent, at least 85 percent, at least 90 percent, at least 95 percent, at least 98 percent, or 100 percent homology or identity to SEQ. ID NO.: 5. The exogenous maltogenic alpha amylase gene from L. plantarum S21 and/or the beta-amylase gene from P. flexa and/or the glucoamylase gene from P. oxalicum are maintained and expressed in a haploid, diploid, or polyploid of the strain of S. cerevisiae. The vector may be expressed in the strain of S. cerevisiae as a single copy or multiple copies.

According to an exemplary embodiment, a method of producing a recombinant yeast strain may comprise the step of integrating an exogenous beta-amylase from P. flexa having at least 80 percent homology to SEQ. ID NO.: 1 and/or an exogenous glucoamylase gene from P. oxalicum having at least 80 percent homology to SEQ. ID NO.: 3 and/or an exogenous maltogenic alpha-amylase gene from L. plantarum S21 having at least 80 percent homology to SEQ. ID NO.: 5 into the genome of a strain of S. cerevisiae.

According to an exemplary embodiment, a vector may comprise an exogenous beta-amylase gene from P. flexa that comprises a sequence having at least 80 percent, at least 85 percent, at least 90 percent, at least 95 percent, at least 98 percent, or 100 percent percent homology or identity to SEQ. ID NO.: 1. The vector may comprise an exogenous glucoamylase gene from P. oxalicum that comprises a sequence having at least 80 percent, at least 85 percent, at least 90 percent, at least 95 percent, at least 98 percent, or 100 percent percent homology or identity to SEQ. ID NO.: 3. The vector may comprise an exogenous maltogenic alpha-amylase gene from L. plantarum S21 that comprises a sequence having at least 80 percent, at least 85 percent, at least 90 percent, at least 95 percent, at least 98 percent, or 100 percent homology or identity to SEQ. ID NO.: 5. The exogenous maltogenic alpha amylase gene from L. plantarum S21 and/or the beta-amylase gene from P. flexa and/or the glucoamylase gene from P. oxalicum are maintained and expressed in a haploid, diploid, or polyploid of the strain of S. cerevisiae. The vector may be expressed in the strain of S. cerevisiae as a single copy or multiple copies.

According to an exemplary embodiment, a method of producing a recombinant yeast strain may comprise the step of integrating an exogenous beta-amylase from P. flexa having at least 80 percent identity to SEQ. ID NO.: 1 and/or an exogenous glucoamylase gene from P. oxalicum having at least 80 percent identity to SEQ. ID NO.: 3 and/or an exogenous maltogenic alpha-amylase gene from L. plantarum S21 having at least 80 percent identity to SEQ. ID NO.: 5 into the genome of a strain of S. cerevisiae.

According to an exemplary embodiment, a yeast strain capable of fermenting corn mash into ethanol with no exogenous glucoamylase and with demonstrable speed and efficiency; the yeast strain may provide for enhanced polysaccharide breakdown along with cofermentation of DP3 sugars, maltose and glucose.

TABLE A
LEGEND

| # | DESCRIPTOR | DEFINITION |
|---|---|---|
| 1 | F15 | Intermediate strain used in the construction of claimed |
| | | inventions |
| 2 | F22 | Maltophilic yeast strain containing beta-amylase, maltogenic |
| | | alpha-amylase and glucoamylase genes |
| 3 | F23 | Maltophilic yeast strain containing beta-amylase and |
| | | glucoamylase genes |
| 4 | T | Terminator |
| 5 | T ADH1 | ADHI gene terminator |
| 6 | T CPS1 | CPS1 gene terminator |
| 7 | T CYC1 | CYCI gene terminator |
| 8 | T PGK1 | PGK1 gene terminator |
| 9 | P | Promoter |
| 10 | P HOR7 | HOR7 gene promoter |
| 11 | P CCW12 | CCW12 gene promoter |
| 12 | SP | Signal Peptide |
| 13 | SP GLM | Signal peptide of GLM protein |
| 14 | SP Ost1-Pro | Signal peptide of Ost1-Pro Alpha factor protein |
| | Alpha factor | |
| 15 | NLS | Neutral Landing Site |
| 16 | NLS7 | Neutral Landing Site Number 7 |
| 17 | NLS9 | Neutral Landing Site Number 9 |
| 18 | NLS3 | Neutral Landing Site Number 3 |
| 19 | SBD | Starch Binding Domain |
| 20 | BAMY | Beta-amylase |
| | Bamy | |
| 21 | BAMY SBD | Beta-amylase fused with Starch Binding Domain |
| 22 | F15-HM- | Maltophilic yeast strain containing beta-amylase and maltogenic |
| | BAMY-78 | alpha-amylase genes |
| 23 | F15-HPoGA- | Maltophilic yeast strain containing glucoamylase and beta- |
| | BAMY-22 | amylase genes |
| 24 | 78-HPoGA- | Maltophilic yeast strain containing beta-amylase, maltogenic |
| | BAMY-49 | alpha-amylase and glucoamylase genes |
| 25 | MA | Maltogenic alpha-amylase |
| 26 | MAL1 | MAL1 gene cluster |
| 27 | MAL2 | MAL2 gene cluster |
| 28 | MAL2-8c | MAL2-8 gene variant encoding a transcription factor protein |
| 29 | PoGA15A | Penicillium oxalicum glucoamylase |
| 30 | NLS-UPS | Neutral Landing Site Upstream Sequence |
| 31 | NLS DWS | Neutral Landing Site Downstream Sequence |
| 32 | NLS2-DWS | Neutral Landing Site Number 2 Downstream Sequence |
| 33 | NotI | DNA Sequence cut by NotI restriction enzyme |
| 34 | ColE1 | ColE1 type plasmid |
| 35 | AmpR | Ampicillin Resistance gene |
| 36 | Chr | Chromosome |
| 37 | Chr XIII | Chromosome 13 |
| 38 | Chr IV | Chromosome 4 |
| 39 | ORF | Open Reading Frame |
| 40 | ER | ETHANOL RED ™ strain of yeast |
| | Ethanol Red | commercially available from Leaf by Lesaffre of Milwaukee, WI |
| | | https://leaf-lesaffre.com/ethanol-innovation/bio-ethanol/ethanol- |
| | | red/ |
| 41 | ER-19-11 | Intermediate strain in the construction of claimed inventions |
| 42 | ER MAL63 | MAL63 gene upstream promoter from ETHANOL RED TM |
| | UPS Promoter | strain of yeast |
| 43 | DP | Degree of polymerization of a starch molecule |
| 44 | DP3 | Starch molecule with 3 linked glucose units |
| 45 | DP4+ | Starch molecule with 4 or more linked glucose units |
| 46 | YDR | Abbreviation for genes found on S. cerevisiae chromosome 4 |
| 47 | YDR436W | Gene encoding a phosphatase protein |
| 48 | YDR439W | Gene encoding a nuceolar protein |
| 49 | YDR440W | Gene encoding a histone methylase protein |
| 50 | YDR443C | Gene encoding a protein that participates in transcription |
| 51 | YCR | Abbreviation for genes found on S. cerevisiae chromosome 3 |
| 52 | YCR022C CDS | Coding sequence of a gene encoding a protein of unknown |
| | | function |
| 53 | YCR023C CDS | Coding sequence of a gene encoding a vacuolar membrane |
| | | protein |
| 54 | HSP30 | Heat Shock 30 gene |
| 55 | ADH3 | Alcohol Dehydrogenase gene 3 |
| 56 | ISF1 | Increasing Suppression Factor gene |
| 57 | pDNLS9-HPCB | Plasmid containing a cassette with HOR7 promoter and |
| | plasmid | PoGA15A gene, CCW12 promoter and Beta-amylase gene that |
| | | can be used for integration into Neutral Landing Site 9 |
| 58 | pDNLS7-HMCB | Plasmid containing a cassette with HOR7 promoter and |
| | plasmid | Maltogenic alpha-amylase gene, CCW12 promoter and Beta- |
| | | amylase gene that can be used for integration into Neutral |
| | | Landing Site 7 |
| 59 | Leading GMO | Recombinant yeast strain widely used in the fuel ethanol |
| | Yeast | industry to ferment corn starch to ethanol |

The embodiments as disclosed and described in the application (including the FIGURES and Examples) are intended to be illustrative and explanatory of the present inventions. Modifications and variations of the disclosed embodiments, including processes employed (or to be employed) and of the compositions and treatments used (or to be used), may be employed; all such modifications and variations are intended to be within the scope of the present inventions.

Protein sequence similarity/identity is calculated by alignment of two protein sequences. Commonly used pairwise alignment tools include COBALT (Papadopoulos and Agarwala, 2007), EMBOSS Needle (Needleman and Wunsch, 1970) and EMBOSS Stretcher (Myers and Miller, 1988). The percentage of identity represents the total fraction of amino acids that are identical along the length of each protein. Similarity is calculated based on the percentage of amino acids with similar character over the reported aligned region. Amino acids are considered similar if they share common chemical properties that impart similar qualities to the structure and activity of the entire protein.

### SEQUENCE LISTING

SEQ ID NO.: 1. CODON OPTIMIZED Beta-amylase (BAMY) FROM P. flexa

| | |
|---|---|
| 1 | agccgtcaac ggccaatcat ttaactcaaa ctataaaact taccttatgg ccccactaaa |
| 61 | aaaagtgact gagttcacta catgggaagc gttcgaaaac gatttgagaa aagcgaagca |
| 121 | aaatggtttt tacgcagtaa cggttgactt ctggtggggt gacatggaga aaaacgggga |
| 181 | tcagcagttc gatttctcat acgcgcagag gtttgcccaa gctgcgagaa acgcaggcat |
| 241 | taaaatggta cctattataa gcacgcatca atgcggaggt aacgtcggcg acgactgcaa |
| 301 | tacgccgcta cctagctgga tttggaacac caagacggat gatagcctat attttaaatc |
| 361 | cgaaacgggc actgtgaaca aagaaaccgt taacccgcta gccacagatg tgataaccaa |
| 421 | gcaatacggt gagctataca ctgcctttgc gcaggcatta gcaccctata aagacgtcat |
| 481 | tccgaaggtc tacctgagcg gagggccagc aggcgagcta aggtatccta gctatacagc |
| 541 | tgccgacgga accggctatc cctctcgtgg aaaatttcaa gcatacaccg actttgccaa |
| 601 | gtccaaattc cagatgtggg cggtaaataa atatggttct ttagccggag tcaaccaggc |
| 661 | gtggggactg agtttgacca gtactagcca gatcctacct ccgagtgatg gtaaccaatt |
| 721 | cttaaaggat ggctataata ctaactacgg caaagacttc ctggaatggt atcagggcgt |
| 781 | attacaggac catgcaaaga ggataggcgc tttagcccat caagcatttg atcctgtctt |
| 841 | caatgtgcct gtcggagcta agatagcagg aattcattgg cagtacaaca accccacaat |
| 901 | gccccacgct gcggaaaagc ccgccggata caataactac agtacactat tagactcttt |
| 961 | taagactgcg aaactggatt taacctttac ctgtctagaa atggttgaca gtggcacgta |
| 1021 | tcccgaatat tccatgccaa aaaccctagt taaagaagtc gctagtcttg cgaacgcgaa |
| 1081 | aggaattgtt cttaatggag agaacgcact tagtataggc agtgaggagc agtacaagcg |
| 1141 | tgccgcggaa atgactttta attacaattt cgcggggttc accttgctaa gattttacga |
| 1201 | cgttataaat aactccacga ggatgtccca attcaatcaa cacctaaaca tcaag |

SEQ ID NO.: 2. PREDICTED PROTEIN PRODUCT OF CODON OPTIMIZED Beta-amylase (BAMY) FROM P. flexa (SEQUENCE NUMBER 1)

| | |
|---|---|
| 1 | avngqsfnsn yktylmaplk kvtefttwea fendlrkakq ngfyavtvdf wwgdmekngd |
| 61 | qqfdfsyaqr faqaarnagi kmvpiisthq cggnvgdden tplpswiwnt ktddslyfks |
| 121 | etgtvnketv nplatdvitk qygelytafa qalapykdvi pkvylsggpa gelrypsyta |
| 181 | adgtgypsrg kfqaytdfak skfqmwavnk ygslagvnqa wglsltstsq ilppsdgnqf |
| 241 | lkdgyntnyg kdflewyqgv lqdhakriga lahqafdpvf nvpvgakiag ihwqynnptm |
| 301 | phaaekpagy nnystlldsf ktakldltft clemvdsgty peysmpktlv kevaslanak |
| 361 | givlngenal sigseeqykr aaemtfnynf agftllrfyd vinnstrmsq fnqhlnik |

SEQ ID NO.: 3. CODON OPTIMIZED GLUCOAMYLASE (POGA) FROM P. oxalicum

| | |
|---|---|
| 1 | gccccacaat tgtcccccag ggctacttct ctagattcct ggttatccag cgaaactact |
| 61 | ttttctttga acggtattct cgccaacatc ggttcttctg gtgcttactc taagtctgct |
| 121 | gcctctggtg ccgtcatcgc ttccccttct actagcaacc ccgattacta ttatacctgg |
| 181 | accagagacg cagcgttaac tttgaaagcc ttagttgata ttttccgtaa tggcaatttg |
| 241 | ggtctacaaa ccgttatcga acaatatgtt aatgcacagg ctaaattgca aactgtctct |
| 301 | aatccttccg gaggtttgtc cgacggtgca ggtttgggag aacctaagtt caatgttgac |
| 361 | ttgtctgctt tcactggtgc ttggggtaga ccacaaagag atggcccggc tctacgggct |
| 421 | atagcactaa tcgatttcgg caattggctg atagataacg gatataaatc ttacgcggtg |
| 481 | aacaacgttt ggccaatcgt aaggaacgat ttggcctatg ttgcccagta ctggtcacag |
| 541 | tccggcttcg acctatggga agaagtgaat tctatgtctt tctttacagt tgctaaccaa |
| 601 | catcgttcat tagtcgaagg atcagctttc gcatctcgtg tcggtgccag ctgttctggt |
| 661 | tgtgactctc aagctcctca gattttgtgt tacatgcaat ctttttggac tgggagttat |
| 721 | attaatgcca atacgggtgg tggtagatcc ggtaaagatt ctaacactat tttagcctcg |
| 781 | atacatactt ttgatcctgc tgcttcttgt gatgacgtta ccttccaacc atgctcaagt |
| 841 | agagctttgg ctaaccacaa ggtctatacc gattctttca gatccgttta cgcgttaaac |
| 901 | tccggtatag cccaaggtaa ggccgtttct gtaggtcgtt acccagaaga tagttactac |
| 961 | ggtggcaacc catggttttt atcaaactta gcagctgctg agcaacttta tgatgctatc |
| 1021 | taccaatgga acaagattgg ttccatcact atcacctcga cctcgcttgc atttttcaag |
| 1081 | gatgtttatc cgtctgccgc taccggtacc tatgcttctg ggtccacaac ctttaatgct |
| 1141 | attatttctg cagtaaagac atatgctgac ggctatgtca gtattgttca atcccactcc |
| 1201 | tatgcgaatg gttcgttgtc agaacaattc gacagaacca ctggtttgtc catcagtgct |
| 1261 | cgcgatttaa catggtctta tgcggcgctg ttgactgcaa atgacagaag aaatggcgtt |
| 1321 | gtccctccat cgtggggcgc aagttccgct aattcgatac ctggttcatg cagcatgggt |
| 1381 | tctgccacag gttcctacgc tactccatct gttggttcat ggccagcaac acttacttca |
| 1441 | ggtacagctg caccttccag tacatcaact actaccaagg ctccaactac caccacggcc |
| 1501 | accacaacaa cttccgccgg ttcctgtact acaccaaccg cagtggctgt tactttcgat |
| 1561 | gaaattgcta cgacgacatt tggtgaaaac gtctacttgg taggaagcat tagccaatta |
| 1621 | ggtaactgga atacagccaa cggtatccca ctgtctgctt caaagtacac ctcttcaaat |
| 1681 | ccattatggt acgccactgt gaacttgccc gctggcacta cttttcaata caaatatttt |
| 1741 | agaaaggaat ctgatggttc catcaaatgg gagtcagacc caaacagatc ttacactgtt |
| 1801 | ccagccaaat gtggtactac tacagccaca gaaaatgata cttggagataa |

SEQ ID NO.: 4. PREDICTED PROTEIN PRODUCT OF CODON OPTIMIZED P. oxalicum (PoGA) (SEQUENCE NUMBER 3)

| | |
|---|---|
| 1 | apqlsprats ldswlssett fslngilani gssgaysksa asgaviasps tsnpdyyytw |
| 61 | trdaaltlka lvdifrngnl glqtvieqyv naqaklqtvs npsgglsdga glgepkfnvd |
| 121 | lsaftgawgr pqrdgpalra ialidfgnwl idngyksyav nnvwpivind layvagywsq |
| 181 | sgfdlweevn smsfftvanq hrslvegsaf asrvgascsg cdsqapqilc ymqsfwtgsy |
| 241 | inantgggrs gkdsntilas ihtfdpaasc ddvtfqpcss ralanhkvyt dsfrsvyaln |
| 301 | sgiaqgkavs vgrypedsyy ggnpwflsnl aaaeqlydai yqwnkigsit itstslaffk |
| 361 | dvypsaatgt yasgsttfna iisavktyad gyvsivqshs yangslseqf drttglsisa |
| 421 | rdltwsyaal ltandrrngv vppswgassa nsipgscsmg satgsyatps vgswpatlts |
| 481 | gtaapsstst ttkaptttta ttttsagsct tptavavtfd eiatttfgen vylvgsisql |
| 541 | gnwntangip lsaskytssn plwyatvnlp agttfqykyf rkesdgsikw esdpnrsytv |
| 601 | pakcgtttat endtwr |

SEQ ID NO.: 5. CODON OPTIMIZED MALTOGENIC ALPHA AMYLASE (MALPS21) FROM L. plantarum S21

| | |
|---|---|
| 1 | gattcataca ccacctcaac agacgattcg tctaatgaca ctgccgacag tgtctctgat |
| 61 | ggtgtgattt tacacgcttg gtgttggtct ttcaacacaa tcaagaacaa tttgaagcaa |
| 121 | attcacgatg caggttacac tgccgttcaa acctcccctg tcaatgaagt caaagttggt |
| 181 | aattctgcta gtaagtcttt gaacaactgg tactggttat accaaccaac aaagtactcg |
| 241 | attggtaact attacttagg taccgaagct gaattcaagt ccatgtgtgc agctgccaag |
| 301 | gagtacaaca tcagaattat tgttgatgct accttgaatg acaccacaag tgactactca |
| 361 | gctatttcgg atgaaatcaa atccattagt aattggactc atggcaatac acagatatcc |
| 421 | aactggtcag acagggagga tgtcacccaa aactctctcc ttggtttgta tgattggaac |
| 481 | actcaaaatt cccaagtcca aacataccta aagaactact tggaacgtct aatatcagat |
| 541 | ggggcaagcg gttttcgtta cgatgcagcc aaacatatcg aattgccatc acaatacgac |
| 601 | ggttcatatg gttccaattt ttggccaaat atcactgaca atggtagtga attccaatat |
| 661 | ggcgaagttt tgcaagattc tatttccaaa gaatccgatt acgctaatta catgtcagta |
| 721 | acagcctcta attatggtaa tactattaga aatgccctga aaaacagaga tttcactgct |
| 781 | agcacattac aaaatttcaa tatttctgtc cccgctagca agttggttac ttgggttgaa |
| 841 | tctcatgaca actatgcaaa cgatgaccaa gtttctacct ggatgaatag ttccgatatt |
| 901 | aaactaggtt gggccgtagt ggcctcaaga tctggaagtg ttccattatt tttcgacaga |
| 961 | ccagttgacg gtggtaatgg tacccgtttt cctggatcta gcgaaattgg tgacgccggt |
| 1021 | tcttcgcttt attatgacaa ggctgttgtg gcggttaaca agttccacaa cgccatggct |
| 1081 | ggtcaatctg aatacatttc aaacccaaac ggtaacacca aaatttttga aaacgaaaga |
| 1141 | ggttctaagg gtgtcgtttt cgctaatgct tcggatggca gctattctct atctgttaag |
| 1201 | acatctcttg ctgacggtac ctacgaaaat aaggccggaa gtgacgagtt cactgttaaa |
| 1261 | aacggttatt tgacaggtac tatccaaggt agagaagtag tcgtattata tggcgatcca |
| 1321 | acttcaagct cgtcctcgtc taccactact gaaactaaga aggtgtattt tgaaaaacca |
| 1381 | tcctcctggg gttccacagt ctatgcctat gtctacaaca aaaacactaa taaggctata |
| 1441 | accagcgcat ggccaggtaa agagatgact gctttaggta atgatgagta taaattagac |
| 1501 | ctggatacag atgaagatga ttccgacttg gcagtaattt tcaccgatgg gaccaaccaa |
| 1561 | actcctgcag ccaacaaggc tgggttcacc ttcacagcag acgcgacgta cgatcagaac |
| 1621 | ggtgttgtta agacctctga ctcatcttcg tcgtcctcca ctaccaccga aacaaaaaaa |
| 1681 | gtgtattttg aaaagccttc atcttggggg tccactgtct acgcctacgt ttataataaa |
| 1741 | aacacgaaca aagctatcac cagtgcttgg cccggtaagg aaatgaccgc tcttggaaat |
| 1801 | gacgaatata aattggattt ggatactgat gaagatgata gtgatctagc tgttatcttt |
| 1861 | actgatggta caaaccaaac gccggcagct aacaaggcag gtttcacttt taccgctgat |
| 1921 | gccacttatg atcaaaacgg tgtggttaag acatctgaca gttcttcatc atcttccagt |
| 1981 | acaactacgg aaactaagaa agtttacttc gaaaagccat cttcgtgggg ctctacggtt |
| 2041 | tacgcttatg tttataacaa gaatacaaat aaagcaatta cttccgcttg gcctggtaag |
| 2101 | gaaatgactg cgttaggcaa cgacgaatac aagttagatt tagataccga tgaagatgat |
| 2161 | agtgatttgg ctgtgatctt cactgatgga accaaccaga ctccagctgc taacaaagca |
| 2221 | ggctttacct ttactgctga tgccacttat gaccagaatg gtgttgtcaa gacctccgat |
| 2281 | agctcctctt cctcgtcaac tactacagaa acgaagaagg tttactttga gaagccaagt |
| 2341 | agttggggtt ctacagttta tgcttacgta tacaataaaa atactaataa agcgatcact |
| 2401 | agcgcctggc caggtaaaga aatgacagct ttgggcaatg acgaatacaa attggacctt |
| 2461 | gacactgacg aggacgactc cgatttggct gttatattta ccgatggtac taatcaaacg |
| 2521 | cctgctgcaa ataaagctgg tttcacattt accgccgatg ctacttacga tcagaacggt |
| 2581 | gtcgtcaaaa catctgattc ttcgtccacc tcttctacat cataa |

SEQ ID NO.: 6. PREDICTED PROTEIN PRODUCT OF CODON OPTIMIZED L. plantarum S21 (MALPS21) (SEQUENCE NUMBER 5)

| | |
|---|---|
| 1 | dsyttstdds sndtadsvsd gvilhawcws fntiknnlkq ihdagytavq tspvnevkvg |
| 61 | nsaskslnnw ywlyqptkys ignyylgtea efksmcaaak eyniriivda tlndttsdys |
| 121 | aisdeiksis nwthgntqis nwsdredvtq nsllglydwn tqnsqvqtyl knylerlisd |
| 181 | gasgfrydaa khielpsqyd gsygsnfwpn itdngsefqy gevlqdsisk esdyanymsv |
| 241 | tasnygntir nalknrdfta stlqnfnisv pasklvtwve shdnyanddq vstwmnssdi |
| 301 | klgwavvasr sgsvplffdr pvdggngtrf pgsseigdag sslyydkavv avnkfhnama |
| 361 | gqseyisnpn gntkifener gskgvvfana sdgsyslsvk tsladgtyen kagsdeftvk |
| 421 | ngyltgtiqg revvvlygdp tssssssttt etkkvyfekp sswgstvyay vynkntnkai |
| 481 | tsawpgkemt algndeykld ldtdeddsdl aviftdgtnq tpaankagft ftadatydqn |
| 541 | gvvktsdsss ssstttetkk vyfekpsswg stvyayvynk ntnkaitsaw pgkemtalgn |
| 601 | deykldldtd eddsdlavif tdgtnqtpaa nkagftftad atydqngvvk tsdsssssss |
| 661 | tttetkkvyf ekpsswgstv yayvynkntn kaitsawpgk emtalgndey kldldtdedd |
| 721 | sdlaviftdg tnqtpaanka gftftadaty dqngvvktsd ssssssttte tkkvyfekps |
| 781 | swgstvyayv ynkntnkait sawpgkemta lgndeykldl dtdeddsdla viftdgtnqt |
| 841 | paankagftf tadatydqng vvktsdssst ssts |

SEQ ID NO.: 7. pDNLS9_HPCB plasmid sequence

### FEATURES Location/Qualifiers

CDS 149 . . . 811/label=“Amp Resistance gene”
misc feature complement (1140 . . . 1147)/label=“Not1”
misc_feature 1151 . . . 1650/label=“UPS_NLS9”
misc_feature 1651 . . . 1658/label=“Pmel”
misc feature 1659 . . . 1848/label-“Terminator CYC1”
misc feature 1849 . . . 1858/label=“Swal site Del”
misc_feature 1859 . . . 2585/label=“Promoter HOR7”
CDS 2586 . . . 2861/label=“Ost1-Pro-a factor signal peptide”
CDS 2862 . . . 4712/label=“PoGA15A”
misc feature 4713 . . . 4718/label=“Apal”
misc_feature 4719 . . . 5146/label=“Terminator PGK1”
misc feature 5147 . . . 5323/label=“CPS1 Terminator”
misc_feature 5324 . . . 5764/label=“Bamy_SBD-An”
CDS complement (5765 . . . 7019)/label=“Bamy”
CDS complement (7019 . . . 7096)/label=“GLM-SP”
misc_feature complement (7097 . . . 7832)/label=“Promoter CCW12”
misc feature 7833 . . . 7840/label=“Swal”
misc feature 7841 . . . 8035/label=“Terminator ADH1”
misc feature 8036 . . . 8043/label=“Pmel”
misc feature 8044 . . . 8543/label=“DWS NLS9”
misc feature 8547 . . . 8554/label=“Not1”
misc_feature 8828 . . . 9456/label=“colEl”

### Origin

| | |
|---|---|
| 1 | cggggtctga cgctcagtgg aacgaaaact cacgttaagg gattttggtc atgagattat |
| 61 | caaaaaggat cttcacctag atccttttaa attaaaaatg aagttttaaa tcaatctaaa |
| 121 | gtatatatga gtaaacttgg tctgacagtt accaatgctt aatcagtgag gcacctatct |
| 181 | cagcgatctg tctatttcgt tcatccatag ttgcctgact ccccgtcgtg tagataacta |
| 241 | cgatacggga gggcttacca tctggcccca gtgctgcaat gataccgcga gacccacgct |
| 301 | caccggctcc agatttatca gcaataaacc agccagccgg aagggccgag cgcagaagtg |
| 361 | gtcctgcaac tttatccgcc tccatccagt ctattaattg ttgccgggaa gctagagtaa |
| 421 | gtagttcgcc agttaatagt ttgcgcaacg ttgttgccat tgctacaggc atcgtggtgt |
| 481 | cacgctcgtc gtttggtatg gcttcattca gctccggttc ccaacgatca aggcgagtta |
| 541 | catgatcccc catgttgtgc aaaaaagcgg ttagctcctt cggtcctccg atcgttgtca |
| 601 | gaagtaagtt ggccgcagtg ttatcactca tggttatggc agcactgcat aattctctta |
| 661 | ctgtcatgcc atccgtaaga tgcttttctg tgactggtga gtactcaacc aagtcattct |
| 721 | gagaatagtg tatgcggcga ccgagttgct cttgcccggc gtcaatacgg gataataccg |
| 781 | cgccacatag cagaacttta aaagtgctca tcattggaaa acgttcttcg gggcgaaaac |
| 841 | tctcaaggat cttaccgctg ttgagatcca gttcgatgta acccactcgt gcacccaact |
| 901 | gatcttcagc atcttttact ttcaccagcg tttctgggtg agcaaaaaca ggaaggcaaa |
| 961 | atgccgcaaa aaagggaata agggcgacac ggaaatgttg aatactcata ctcttccttt |
| 1021 | ttcaatatta ttgaagcatt tatcagggtt attgtctcat gagcggatac atatttgaat |
| 1081 | gtatttagaa aaataaacaa ataggggttc cgcgcacatt tccccgaaaa gtgccacctg |
| 1141 | cggccgcatc ctgacatttt gttgttttcc ttgcgtgccc aatgttgcca gcgtaaaagt |
| 1201 | tatcgtaaac gttatcgtaa acgttatccc cgttttgtaa actttattgt cttcgtaaaa |
| 1261 | aatagaaacc aacacttaaa attggtccca taaaaaattg agtcagttcc ttcatcgtgg |
| 1321 | tacagtcaga gtattctaaa ttaattaatt tactggtaat ttaataacat gaagaagaaa |
| 1381 | gattggatat tgaagttctc caccaggcac atataacgct ctaaataata tttagatgtt |
| 1441 | gcactaaagt ccacgatgaa gaataaaaag catcacttga tcgttcaatg agccaatctt |
| 1501 | ggaaattttg acacctctat taaccagaac atccagcttg ttgagaactg taactttgaa |
| 1561 | actcaaataa ctcaaggaac gggagtcttt caagattttg gaataatttg accactttgt |
| 1621 | ttttaaacaa ttcgagtgaa ggccatgcgc gtttaaacct tcgagcgtcc caaaaccttc |
| 1681 | tcaagcaagg ttttcagtat aatgttacat gcgtacacgc gtttgtacag aaaaaaaaga |
| 1741 | aaaatttgaa atataaataa cgttcttaat actaacataa ctataaaaaa ataaataggg |
| 1801 | acctagactt caggttgtct aactccttcc ttttcggtta gagcggatat ttcgaaatct |
| 1861 | ttcgattagc acgcacacac atcacataga ctgcgtcata aaaatacact acggaaaaac |
| 1921 | cataaagagc aaagcgatac ctacttggaa ggaaaaggag cacgcttgta agggggatgg |
| 1981 | gggctaagaa gtcattcact ttcttttccc ttcgcggtcc ggacccggga cccctcctct |
| 2041 | ccccgcacaa tttcttcctt tcatatcttc cttttattcc tatcccgttg aagcaaccgc |
| 2101 | actatgacta aatggtgctg gacatctcca tggctgtgac ttgtgtgtat ctcacagtgg |
| 2161 | taacggcacc gtggctcgga aacggttcct tcgtgacaat tctagaacag gggctacagt |
| 2221 | ctcgataata gaataataag cgcatttttg ttagcgccgc cgcggcgccc gtttcccaat |
| 2281 | agggaggcgc agtttatcgg cggagcttta cttcttccta tttgggtaag cccctttctg |
| 2341 | ttttcggcca gtggttgctg caggctgcgc cggagaacat agtgataagg gatgtaactt |
| 2401 | tcgatgagag aattagcaag cggaaaaaaa actatggcta gctgggagtt gtttttcaat |
| 2461 | catataaaag ggagaaattg ttgctcacta tgtgacagtt tctgggacgt cttaactttt |
| 2521 | attgcagagg actatcaaat catacagata ttgtcaaaaa aaaaaaaaaa gactaataat |
| 2581 | aaaaaatgag acaagtctgg ttctcttgga ttgttggttt attcttatgt ttttttaacg |
| 2641 | tttcgtcagc tgctccagtt aacactacaa ctgaagacga gaccgctcaa attccagctg |
| 2701 | aggcagtcat tggttacagt gatttggaag gtgactttga cgtggcagtt ttaccattta |
| 2761 | gtaactctac caataatggg ttgctattca ttaatactac catcgcctca attgctgcta |
| 2821 | aggaggaagg tgttagcttg gataaaaggg aagccgaagc tgccccacaa ttgtccccca |
| 2881 | gggctacttc tctagattcc tggttatcca gcgaaactac tttttctttg aacggtattc |
| 2941 | tcgccaacat cggttcttct ggtgcttact ctaagtctgc tgcctctggt gccgtcatcg |
| 3001 | cttccccttc tactagcaac cccgattact attatacctg gaccagagac gcagcgttaa |
| 3061 | ctttgaaagc cttagttgat attttccgta atggcaattt gggtctacaa accgttatcg |
| 3121 | aacaatatgt taatgcacag gctaaattgc aaactgtctc taatccttcc ggaggtttgt |
| 3181 | ccgacggtgc aggtttggga gaacctaagt tcaatgttga cttgtctgct ttcactggtg |
| 3241 | cttggggtag accacaaaga gatggcccgg ctctacgggc tatagcacta atcgatttcg |
| 3301 | gcaattggct gatagataac ggatataaat cttacgcggt gaacaacgtt tggccaatcg |
| 3361 | taaggaacga tttggcctat gttgcccagt actggtcaca gtccggcttc gacctatggg |
| 3421 | aagaagtgaa ttctatgtct ttctttacag ttgctaacca acatcgttca ttagtcgaag |
| 3481 | gatcagcttt cgcatctcgt gtcggtgcca gctgttctgg ttgtgactct caagctcctc |
| 3541 | agattttgtg ttacatgcaa tctttttgga ctgggagtta tattaatgcc aatacgggtg |
| 3601 | gtggtagatc cggtaaagat tctaacacta ttttagcctc gatacatact tttgatcctg |
| 3661 | ctgcttcttg tgatgacgtt accttccaac catgctcaag tagagctttg gctaaccaca |
| 3721 | aggtctatac cgattctttc agatccgttt acgcgttaaa ctccggtata gcccaaggta |
| 3781 | aggccgtttc tgtaggtcgt tacccagaag atagttacta cggtggcaac ccatggtttt |
| 3841 | tatcaaactt agcagctgct gagcaacttt atgatgctat ctaccaatgg aacaagattg |
| 3901 | gttccatcac tatcacctcg acctcgcttg catttttcaa ggatgtttat ccgtctgccg |
| 3961 | ctaccggtac ctatgcttct gggtccacaa cctttaatgc tattatttct gcagtaaaga |
| 4021 | catatgctga cggctatgtc agtattgttc aatcccactc ctatgcgaat ggttcgttgt |
| 4081 | cagaacaatt cgacagaacc actggtttgt ccatcagtgc tcgcgattta acatggtctt |
| 4141 | atgcggcgct gttgactgca aatgacagaa gaaatggcgt tgtccctcca tcgtggggcg |
| 4201 | caagttccgc taattcgata cctggttcat gcagcatggg ttctgccaca ggttcctacg |
| 4261 | ctactccatc tgttggttca tggccagcaa cacttacttc aggtacagct gcaccttcca |
| 4321 | gtacatcaac tactaccaag gctccaacta ccaccacggc caccacaaca acttccgccg |
| 4381 | gttcctgtac tacaccaacc gcagtggctg ttactttcga tgaaattgct acgacgacat |
| 4441 | ttggtgaaaa cgtctacttg gtaggaagca ttagccaatt aggtaactgg aatacagcca |
| 4501 | acggtatccc actgtctgct tcaaagtaca cctcttcaaa tccattatgg tacgccactg |
| 4561 | tgaacttgcc cgctggcact acttttcaat acaaatattt tagaaaggaa tctgatggtt |
| 4621 | ccatcaaatg ggagtcagac ccaaacagat cttacactgt tccagccaaa tgtggtacta |
| 4681 | ctacagccac agaaaatgat acttggagat aagggcccat tgaattgaat tgaaatcgat |
| 4741 | agatcaattt ttttcttttc tctttcccca tcctttacgc taaaataata gtttatttta |
| 4801 | ttttttgaat attttttatt tatatacgta tatatagact attatttatc ttttaatgat |
| 4861 | tattaagatt tttattaaaa aaaaattcgc tcctctttta atgcctttat ccagtttttt |
| 4921 | tttcccattc gatatttcta tgttcgggtt cagcgtattt taagtttaat aactcgaaaa |
| 4981 | ttctgcgttc gttaaagctt tcgagaagga tattatttcg aaataaaccg tgttgtgtaa |
| 5041 | gcttgaagcc tttttgcgct gccaatattc ttatccatct attgtactct ttagatccag |
| 5101 | tatagtgtat tcttcctgct ccaagttcat cccacttgca acaaaaattt gacacttgat |
| 5161 | ttgacacttc tttttttttt tatttatgtt ttgtttaact gacggtgttt ttttttttta |
| 5221 | cgttatactg atacatatat agacgtgatt tatttaaata aaaagttcta tgaaaaaaaa |
| 5281 | aaaaaattaa aaaaaaaaat ctttgactat tcaatcattg cgcttacctc caagtgtctg |
| 5341 | taaccgtggc cgtggaggtc ccgcatgcct gcggaaccgt atattctctg tttgggtcag |
| 5401 | attcccactc aacagagtcg tcagattcga tcctaataaa tttatattcg aaggactcac |
| 5461 | ctgcgggtag tgtgacggtg acataccaaa gagggtcaga actggtgtac ttatctgcag |
| 5521 | acagagcaat cccatcagag gtttcccaat cccccaactg agagatactc ccgacaagat |
| 5581 | agatgttttc tccgtaggta gtggtggccg ttaggtcgaa tgtgacagca acagcggtcg |
| 5641 | gagtagtgca tgaagtgctt gatgtggaag ttgatgtttt gctggctgtt gcagtcgtct |
| 5701 | tagacgttga cgttacgcta cctgatcctg tcggagtcgc ggttgttgtc gtgccgccgg |
| 5761 | tggccttgat gtttaggtgt tgattgaatt gggacatcct cgtggagtta tttataacgt |
| 5821 | cgtaaaatct tagcaaggtg aaccccgcga aattgtaatt aaaagtcatt tccgcggcac |
| 5881 | gcttgtactg ctcctcactg cctatactaa gtgcgttctc tccattaaga acaattcctt |
| 5941 | tcgcgttcgc aagactagcg acttctttaa ctagggtttt tggcatggaa tattcgggat |
| 6001 | acgtgccact gtcaaccatt tctagacagg taaaggttaa atccagtttc gcagtcttaa |
| 6061 | aagagtctaa tagtgtactg tagttattgt atccggcggg cttttccgca gcgtggggca |
| 6121 | ttgtggggtt gttgtactgc caatgaattc ctgctatctt agctccgaca ggcacattga |
| 6181 | agacaggatc aaatgcttga tgggctaaag cgcctatcct ctttgcatgg tcctgtaata |
| 6241 | cgccctgata ccattccagg aagtctttgc cgtagttagt attatagcca tcctttaaga |
| 6301 | attggttacc atcactcgga ggtaggatct ggctagtact ggtcaaactc agtccccacg |
| 6361 | cctggttgac tccggctaaa gaaccatatt tatttaccgc ccacatctgg aatttggact |
| 6421 | tggcaaagtc ggtgtatgct tgaaattttc cacgagaggg atagccggtt ccgtcggcag |
| 6481 | ctgtatagct aggatacctt agctcgcctg ctggccctcc gctcaggtag accttcggaa |
| 6541 | tgacgtcttt atagggtgct aatgcctgcg caaaggcagt gtatagctca ccgtattgct |
| 6601 | tggttatcac atctgtggct agcgggttaa cggtttcttt gttcacagtg cccgtttcgg |
| 6661 | atttaaaata taggctatca tccgtcttgg tgttccaaat ccagctaggt agcggcgtat |
| 6721 | tgcagtcgtc gccgacgtta cctccgcatt gatgcgtgct tataataggt accattttaa |
| 6781 | tgcctgcgtt tctcgcagct tgggcaaacc tctgcgcgta tgagaaatcg aactgctgat |
| 6841 | ccccgttttt ctccatgtca ccccaccaga agtcaaccgt tactgcgtaa aaaccatttt |
| 6901 | gcttcgcttt tctcaaatcg ttttcgaacg cttcccatgt agtgaactca gtcacttttt |
| 6961 | ttagtggggc cataaggtaa gttttatagt ttgagttaaa tgattggccg ttgacggctc |
| 7021 | tcttgtcaag ctcaacggga acacaagatg cgacagctgc gaaaacagcg gttaagaaga |
| 7081 | cagtcaatct gatcattatt gatatagtgt ttaagcgaat gacagaagat taatttcttg |
| 7141 | gtatgttagg aaagaataaa ggagaataag aataattaga acaatgtagg atggaaagaa |
| 7201 | agattatcaa gcatgccgac tttatatact tgaacggagg caaaggatgc aaaattttct |
| 7261 | cacatttctt tctgccgtta tgttggaagt aagactccca ttatcgcaat actgcaacac |
| 7321 | gaatatgcaa aatttgctga gttatcgcag atagttgttg caaagatagc ggcgtaggtg |
| 7381 | gccgcgaaat ggggaattcc aaaacaaacg gtttttttac tcctgagaaa tacttgtacg |
| 7441 | ggataatcca gggcctacca cccacgcttc gaggattggc ttttattttt ttttttttgg |
| 7501 | tggcgtttta tttctttccc gctttctggg acttgtgcgg agttttgaga ggggcgcgcg |
| 7561 | gcaaaggatt cccaaaacgg aaatcagacg ccaatagcca gcactcaaag cagttctgga |
| 7621 | cccattgcga ttttcccatt tggttcttgc gcgtgctgat tccgacacgc gcgtctataa |
| 7681 | atagcatgaa gtatccgcac accgcagcgt tagtgaggtg aggggtgcag caagctaatt |
| 7741 | cccgcatctg gaatctgaac tgcccctttt ggactaaccg tgtggttcat gggtgggcga |
| 7801 | agtgcgcaac ctcgaaggtt ttcttttgcg tgatttaaat gtagatacgt tgttgacact |
| 7861 | tctaaataag cgaatttctt atgatttatg atttttatta ttaaataagt tataaaaaaa |
| 7921 | ataagtgtat acaaatttta aagtgactct taggttttaa aacgaaaatt cttattcttg |
| 7981 | agtaactctt tcctgtaggt caggttgctt tctcaggtat agcatgaggt cgctcgttta |
| 8041 | aacttaaaaa catttctttc tttatccaat tgaattatgc cattcgatat taaatacgac |
| 8101 | aacaggaaca ctgaagaacg ggatactccc cccttgtgaa tgtgctacaa taaacagaat |
| 8161 | atcggcaccc ggttatttct tctctccaat tcttactaaa ttagcgataa attctaccct |
| 8221 | ttccataacg ggccgttctt ctgcaagaga atttttccta cttgaatagg catatttgaa |
| 8281 | tctgaaaagt agtcacagat agtgaactct gctttattaa taaaattcca ggatgtactc |
| 8341 | ttggagatcg aatcaagttc ctagaaatcc tttgagaaag aagcggtgaa tacccgtagg |
| 8401 | taggattttt tcacgtaatt aaaccgcctt atcttatttg ttgccagaaa actagaggat |
| 8461 | ggaataaaca tgttccctaa tggtgtggca tttacctgtc tgtctagttt ctgttagtgc |
| 8521 | attccaatgt cagctgcttc gcttatgcgg ccgcggatct gccggtctcc ctatagtgag |
| 8581 | tcgtattaat ttcgataagc caggttaacc tgcattaatg aatcggccaa cgcgcgggga |
| 8641 | gaggcggttt gcgtattggg cgctcttccg cttcctcgct cactgactcg ctgcgctcgg |
| 8701 | tcgttcggct gcggcgagcg gtatcagctc actcaaaggc ggtaatacgg ttatccacag |
| 8761 | aatcagggga taacgcagga aagaacatgt gagcaaaagg ccagcaaaag gccaggaacc |
| 8821 | gtaaaaaggc cgcgttgctg gcgtttttcc ataggctccg cccccctgac gagcatcaca |
| 8881 | aaaatcgacg ctcaagtcag aggtggcgaa acccgacagg actataaaga taccaggcgt |
| 8941 | ttccccctgg aagctccctc gtgcgctctc ctgttccgac cctgccgctt accggatacc |
| 9001 | tgtccgcctt tctcccttcg ggaagcgtgg cgctttctca atgctcacgc tgtaggtatc |
| 9061 | tcagttcggt gtaggtcgtt cgctccaagc tgggctgtgt gcacgaaccc cccgttcagc |
| 9121 | ccgaccgctg cgccttatcc ggtaactatc gtcttgagtc caacccggta agacacgact |
| 9181 | tatcgccact ggcagcagcc actggtaaca ggattagcag agcgaggtat gtaggcggtg |
| 9241 | ctacagagtt cttgaagtgg tggcctaact acggctacac tagaaggaca gtatttggta |
| 9301 | tctgcgctct gctgaagcca gttaccttcg gaaaaagagt tggtagctct tgatccggca |
| 9361 | aacaaaccac cgctggtagc ggtggttttt ttgtttgcaa gcagcagatt acgcgcagaa |
| 9421 | aaaaaggatc tcaagaagat cctttgatct tttcta |

SEQ ID NO.: 8. pDNLS7_HMCB plasmid sequence

### FEATURES Location/Qualifiers

CDS 149 . . . 811/label=“Amp Resistance gene”
misc_feature complement (1140 . . . 1147)/label=“Not1”
misc_feature 1151 . . . 1650/label=“UPS NLS7”
misc_feature 1651 . . . 1658/label=“Pmel”
misc feature 1659 . . . 1850/label=“Terminator CYC1”
misc_feature 1851 . . . 1860/label=“Swal site del”
misc feature 1861 . . . 2587/label=“Promoter HOR7”
CDS 2588 . . . 2665/label=“GLM-SP”
CDS 2666 . . . 5290/label=“MALPS21”
misc_feature 5291 . . . 5718/label=“Terminator PGK1”
misc_feature 5719 . . . 5724/label=“Apal”
misc_feature 5725 . . . 5901/label=“CPS1 Terminator”
misc feature 5902 . . . 6342/label=“Bamy_SBD-An”
CDS complement (6343 . . . 7597)/label=“Bamy”
CDS complement (7597 . . . 7674)/label-“GLM-SP”
misc feature complement (7675 . . . 8410)/label=“Promoter CCW12”
misc feature 8411 . . . 8416/label=“Apal”
misc feature 8420 . . . 8427/label=“Swal”
misc_feature 8428 . . . 8622/label=“Terminator ADH1”
misc feature 8623 . . . 8630/label=“Pmel”
misc_feature 8631 . . . 9130/label=“DWS NLS7”
misc_feature 9134 . . . 9141/label=“Not1”
misc feature 9415 . . . 10043/label=“colEl”

### Origin

| | |
|---|---|
| 1 | cggggtctga cgctcagtgg aacgaaaact cacgttaagg gattttggtc atgagattat |
| 61 | caaaaaggat cttcacctag atccttttaa attaaaaatg aagttttaaa tcaatctaaa |
| 121 | gtatatatga gtaaacttgg tctgacagtt accaatgctt aatcagtgag gcacctatct |
| 181 | cagcgatctg tctatttcgt tcatccatag ttgcctgact ccccgtcgtg tagataacta |
| 241 | cgatacggga gggcttacca tctggcccca gtgctgcaat gataccgcga gacccacgct |
| 301 | caccggctcc agatttatca gcaataaacc agccagccgg aagggccgag cgcagaagtg |
| 361 | gtcctgcaac tttatccgcc tccatccagt ctattaattg ttgccgggaa gctagagtaa |
| 421 | gtagttcgcc agttaatagt ttgcgcaacg ttgttgccat tgctacaggc atcgtggtgt |
| 481 | cacgctcgtc gtttggtatg gcttcattca gctccggttc ccaacgatca aggcgagtta |
| 541 | catgatcccc catgttgtgc aaaaaagcgg ttagctcctt cggtcctccg atcgttgtca |
| 601 | gaagtaagtt ggccgcagtg ttatcactca tggttatggc agcactgcat aattctctta |
| 661 | ctgtcatgcc atccgtaaga tgcttttctg tgactggtga gtactcaacc aagtcattct |
| 721 | gagaatagtg tatgcggcga ccgagttgct cttgcccggc gtcaatacgg gataataccg |
| 781 | cgccacatag cagaacttta aaagtgctca tcattggaaa acgttcttcg ggggaaaac |
| 841 | tctcaaggat cttaccgctg ttgagatcca gttcgatgta acccactcgt gcacccaact |
| 901 | gatcttcagc atcttttact ttcaccagcg tttctgggtg agcaaaaaca ggaaggcaaa |
| 961 | atgccgcaaa aaagggaata agggcgacac ggaaatgttg aatactcata ctcttccttt |
| 1021 | ttcaatatta ttgaagcatt tatcagggtt attgtctcat gagcggatac atatttgaat |
| 1081 | gtatttagaa aaataaacaa ataggggttc cgcgcacatt tccccgaaaa gtgccacctg |
| 1141 | cggccgcatc ccattttgag cgagagaacc catttttcta tacaaatttc actagagcac |
| 1201 | ggccgttaca tttagtaata gccaataagg gttttttatc gattagtgtt ccctgcgctc |
| 1261 | cttaacatca tacaaccgag tccttgacat ggaaatagta ggcaagtaaa ccaaagtcct |
| 1321 | ttcttcaaaa gtagaaaact tgagcactta tttcctgcgc atgtcatatg ttaattttcc |
| 1381 | ttaactgcgc tgaatacgtc ctgtcaattc aaatatatca cgttttgagc agccctaaag |
| 1441 | aagaaaacct caacagcagt attactatta caatcaaaca actttagtgc cgcgtgatac |
| 1501 | cgggggttga agtgggtgca ttgagccgta ttcttcttcc ccgtaagaaa gttatgtatc |
| 1561 | ctttttactg cgttgtaata gcttctgaaa acctaaaaaa tgaacgctat gtagctcata |
| 1621 | tccgtttcgc ataagtaaga ataactactt gtttaaacct tcgagcgtcc caaaaccttc |
| 1681 | tcaagcaagg ttttcagtat aatgttacat gcgtacacgc gtttgtacag aaaaaaaaga |
| 1741 | aaaatttgaa atataaataa cgttcttaat actaacataa ctattaaaaa aaataaatag |
| 1801 | ggacctagac ttcaggttgt ctaactcctt ccttttcggt tagagcggat atttcgaaat |
| 1861 | ctttcgatta gcacgcacac acatcacata gactgcgtca taaaaataca ctacggaaaa |
| 1921 | accataaaga gcaaagcgat acctacttgg aaggaaaagg agcacgcttg taagggggat |
| 1981 | gggggctaag aagtcattca ctttcttttc ccttcgcggt ccggacccgg gacccctcct |
| 2041 | ctccccgcac aatttcttcc tttcatatct tccttttatt cctatcccgt tgaagcaacc |
| 2101 | gcactatgac taaatggtgc tggacatctc catggctgtg acttgtgtgt atctcacagt |
| 2161 | ggtaacggca ccgtggctcg gaaacggttc cttcgtgaca attctagaac aggggctaca |
| 2221 | gtctcgataa tagaataata agcgcatttt tgttagcgcc gccgcggcgc ccgtttccca |
| 2281 | atagggaggc gcagtttatc ggcggagctt tacttcttcc tatttgggta agcccctttc |
| 2341 | tgttttcggc cagtggttgc tgcaggctgc gccggagaac atagtgataa gggatgtaac |
| 2401 | tttcgatgag agaattagca agcggaaaaa aaactatggc tagctgggag ttgtttttca |
| 2461 | atcatataaa agggagaaat tgttgctcac tatgtgacag tttctgggac gtcttaactt |
| 2521 | ttattgcaga ggactatcaa atcatacaga tattgtcaaa aaaaaaaaaa aagactaata |
| 2581 | ataaaaaatg atcagattga ctgtcttctt aaccgctgtt ttcgcagctg tcgcatcttg |
| 2641 | tgttcccgtt gagcttgaca agagagattc atacaccacc tcaacagacg attcgtctaa |
| 2701 | tgacactgcc gacagtgtct ctgatggtgt gattttacac gcttggtgtt ggtctttcaa |
| 2761 | cacaatcaag aacaatttga agcaaattca cgatgcaggt tacactgccg ttcaaacctc |
| 2821 | ccctgtcaat gaagtcaaag ttggtaattc tgctagtaag tctttgaaca actggtactg |
| 2881 | gttataccaa ccaacaaagt actcgattgg taactattac ttaggtaccg aagctgaatt |
| 2941 | caagtccatg tgtgcagctg ccaaggagta caacatcaga attattgttg atgctacctt |
| 3001 | gaatgacacc acaagtgact actcagctat ttcggatgaa atcaaatcca ttagtaattg |
| 3061 | gactcatggc aatacacaga tatccaactg gtcagacagg gaggatgtca cccaaaactc |
| 3121 | tctccttggt ttgtatgatt ggaacactca aaattcccaa gtccaaacat acctaaagaa |
| 3181 | ctacttggaa cgtctaatat cagatggggc aagcggtttt cgttacgatg cagccaaaca |
| 3241 | tatcgaattg ccatcacaat acgacggttc atatggttcc aatttttggc caaatatcac |
| 3301 | tgacaatggt agtgaattcc aatatggcga agttttgcaa gattctattt ccaaagaatc |
| 3361 | cgattacgct aattacatgt cagtaacagc ctctaattat ggtaatacta ttagaaatgc |
| 3421 | cctgaaaaac agagatttca ctgctagcac attacaaaat ttcaatattt ctgtccccgc |
| 3481 | tagcaagttg gttacttggg ttgaatctca tgacaactat gcaaacgatg accaagtttc |
| 3541 | tacctggatg aatagttccg atattaaact aggttgggcc gtagtggcct caagatctgg |
| 3601 | aagtgttcca ttatttttcg acagaccagt tgacggtggt aatggtaccc gttttcctgg |
| 3661 | atctagcgaa attggtgacg ccggttcttc gctttattat gacaaggctg ttgtggcggt |
| 3721 | taacaagttc cacaacgcca tggctggtca atctgaatac atttcaaacc caaacggtaa |
| 3781 | caccaaaatt tttgaaaacg aaagaggttc taagggtgtc gttttcgcta atgcttcgga |
| 3841 | tggcagctat tctctatctg ttaagacatc tcttgctgac ggtacctacg aaaataaggc |
| 3901 | cggaagtgac gagttcactg ttaaaaacgg ttatttgaca ggtactatcc aaggtagaga |
| 3961 | agtagtcgta ttatatggcg atccaacttc aagctcgtcc tcgtctacca ctactgaaac |
| 4021 | taagaaggtg tattttgaaa aaccatcctc ctggggttcc acagtctatg cctatgtcta |
| 4081 | caacaaaaac actaataagg ctataaccag cgcatggcca ggtaaagaga tgactgcttt |
| 4141 | aggtaatgat gagtataaat tagacctgga tacagatgaa gatgattccg acttggcagt |
| 4201 | aattttcacc gatgggacca accaaactcc tgcagccaac aaggctgggt tcaccttcac |
| 4261 | agcagacgcg acgtacgatc agaacggtgt tgttaagacc tctgactcat cttcgtcgtc |
| 4321 | ctccactacc accgaaacaa aaaaagtgta ttttgaaaag ccttcatctt gggggtccac |
| 4381 | tgtctacgcc tacgtttata ataaaaacac gaacaaagct atcaccagtg cttggcccgg |
| 4441 | taaggaaatg accgctcttg gaaatgacga atataaattg gatttggata ctgatgaaga |
| 4501 | tgatagtgat ctagctgtta tctttactga tggtacaaac caaacgccgg cagctaacaa |
| 4561 | ggcaggtttc acttttaccg ctgatgccac ttatgatcaa aacggtgtgg ttaagacatc |
| 4621 | tgacagttct tcatcatctt ccagtacaac tacggaaact aagaaagttt acttcgaaaa |
| 4681 | gccatcttcg tggggctcta cggtttacgc ttatgtttat aacaagaata caaataaagc |
| 4741 | aattacttcc gcttggcctg gtaaggaaat gactgcgtta ggcaacgacg aatacaagtt |
| 4801 | agatttagat accgatgaag atgatagtga tttggctgtg atcttcactg atggaaccaa |
| 4861 | ccagactcca gctgctaaca aagcaggctt tacctttact gctgatgcca cttatgacca |
| 4921 | gaatggtgtt gtcaagacct ccgatagctc ctcttcctcg tcaactacta cagaaacgaa |
| 4981 | gaaggtttac tttgagaagc caagtagttg gggttctaca gtttatgctt acgtatacaa |
| 5041 | taaaaatact aataaagcga tcactagcgc ctggccaggt aaagaaatga cagctttggg |
| 5101 | caatgacgaa tacaaattgg accttgacac tgacgaggac gactccgatt tggctgttat |
| 5161 | atttaccgat ggtactaatc aaacgcctgc tgcaaataaa gctggtttca catttaccgc |
| 5221 | cgatgctact tacgatcaga acggtgtcgt caaaacatct gattcttcgt ccacctcttc |
| 5281 | tacatcataa attgaattga attgaaatcg atagatcaat ttttttcttt tctctttccc |
| 5341 | catcctttac gctaaaataa tagtttattt tattttttga atatttttta tttatatacg |
| 5401 | tatatataga ctattattta tcttttaatg attattaaga tttttattaa aaaaaaattc |
| 5461 | gctcctcttt taatgccttt atccagtttt tttttcccat tcgatatttc tatgttcggg |
| 5521 | ttcagcgtat tttaagttta ataactcgaa aattctgcgt tcgttaaagc tttcgagaag |
| 5581 | gatattattt cgaaataaac cgtgttgtgt aagcttgaag cctttttgcg ctgccaatat |
| 5641 | tcttatccat ctattgtact ctttagatcc agtatagtgt attcttcctg ctccaagttc |
| 5701 | atcccacttg caacaaaagg gcccatttga cacttgattt gacacttctt ttttttttta |
| 5761 | tttatgtttt gtttaactga cggtgttttt tttttttacg ttatactgat acatatatag |
| 5821 | acgtgattta tttaaataaa aagttctatg aaaaaaaaaa aaaattaaaa aaaaaaatct |
| 5881 | ttgactattc aatcattgcg cttacctcca agtgtctgta accgtggccg tggaggtccc |
| 5941 | gcatgcctgc ggaaccgtat attctctgtt tgggtcagat tcccactcaa cagagtcgtc |
| 6001 | agattcgatc ctaataaatt tatattcgaa ggactcacct gcgggtagtg tgacggtgac |
| 6061 | ataccaaaga gggtcagaac tggtgtactt atctgcagac agagcaatcc catcagaggt |
| 6121 | ttcccaatcc cccaactgag agatactccc gacaagatag atgttttctc cgtaggtagt |
| 6181 | ggtggccgtt aggtcgaatg tgacagcaac agcggtcgga gtagtgcatg aagtgcttga |
| 6241 | tgtggaagtt gatgttttgc tggctgttgc agtcgtctta gacgttgacg ttacgctacc |
| 6301 | tgatcctgtc ggagtcgcgg ttgttgtcgt gccgccggtg gccttgatgt ttaggtgttg |
| 6361 | attgaattgg gacatcctcg tggagttatt tataacgtcg taaaatctta gcaaggtgaa |
| 6421 | ccccgcgaaa ttgtaattaa aagtcatttc cgcggcacgc ttgtactgct cctcactgcc |
| 6481 | tatactaagt gcgttctctc cattaagaac aattcctttc gcgttcgcaa gactagcgac |
| 6541 | ttctttaact agggtttttg gcatggaata ttcgggatac gtgccactgt caaccatttc |
| 6601 | tagacaggta aaggttaaat ccagtttcgc agtcttaaaa gagtctaata gtgtactgta |
| 6661 | gttattgtat ccggcgggct tttccgcagc gtggggcatt gtggggttgt tgtactgcca |
| 6721 | atgaattcct gctatcttag ctccgacagg cacattgaag acaggatcaa atgcttgatg |
| 6781 | ggctaaagcg cctatcctct ttgcatggtc ctgtaatacg ccctgatacc attccaggaa |
| 6841 | gtctttgccg tagttagtat tatagccatc ctttaagaat tggttaccat cactcggagg |
| 6901 | taggatctgg ctagtactgg tcaaactcag tccccacgcc tggttgactc cggctaaaga |
| 6961 | accatattta tttaccgccc acatctggaa tttggacttg gcaaagtcgg tgtatgcttg |
| 7021 | aaattttcca cgagagggat agccggttcc gtcggcagct gtatagctag gataccttag |
| 7081 | ctcgcctgct ggccctccgc tcaggtagac cttcggaatg acgtctttat agggtgctaa |
| 7141 | tgcctgcgca aaggcagtgt atagctcacc gtattgcttg gttatcacat ctgtggctag |
| 7201 | cgggttaacg gtttctttgt tcacagtgcc cgtttcggat ttaaaatata ggctatcatc |
| 7261 | cgtcttggtg ttccaaatcc agctaggtag cggcgtattg cagtcgtcgc cgacgttacc |
| 7321 | tccgcattga tgcgtgctta taataggtac cattttaatg cctgcgtttc tcgcagcttg |
| 7381 | ggcaaacctc tgcgcgtatg agaaatcgaa ctgctgatcc ccgtttttct ccatgtcacc |
| 7441 | ccaccagaag tcaaccgtta ctgcgtaaaa accattttgc ttcgcttttc tcaaatcgtt |
| 7501 | ttcgaacgct tcccatgtag tgaactcagt cacttttttt agtggggcca taaggtaagt |
| 7561 | tttatagttt gagttaaatg attggccgtt gacggctctc ttgtcaagct caacgggaac |
| 7621 | acaagatgcg acagctgcga aaacagcggt taagaagaca gtcaatctga tcattattga |
| 7681 | tatagtgttt aagcgaatga cagaagatta atttcttggt atgttaggaa agaataaagg |
| 7741 | agaataagaa taattagaac aatgtaggat ggaaagaaag attatcaagc atgccgactt |
| 7801 | tatatacttg aacggaggca aaggatgcaa aattttctca catttctttc tgccgttatg |
| 7861 | ttggaagtaa gactcccatt atcgcaatac tgcaacacga atatgcaaaa tttgctgagt |
| 7921 | tatcgcagat agttgttgca aagatagcgg cgtaggtggc cgcgaaatgg ggaattccaa |
| 7981 | aacaaacggt ttttttactc ctgagaaata cttgtacggg ataatccagg gcctaccacc |
| 8041 | cacgcttcga ggattggctt ttattttttt ttttttggtg gcgttttatt tctttcccgc |
| 8101 | tttctgggac ttgtgcggag ttttgagagg ggcgcgcggc aaaggattcc caaaacggaa |
| 8161 | atcagacgcc aatagccagc actcaaagca gttctggacc cattgcgatt ttcccatttg |
| 8221 | gttcttgcgc gtgctgattc cgacacgcgc gtctataaat agcatgaagt atccgcacac |
| 8281 | cgcagcgtta gtgaggtgag ggtggcagca agctaattcc cgcatctgga atctgaactg |
| 8341 | ccccttttgg actaaccgtg tggttcatgg gtgggcgaag tgcgcaacct cgaaggtttt |
| 8401 | cttttgcgtg gggccctata tttaaatgta gatacgttgt tgacacttct aaataagcga |
| 8461 | atttcttatg atttatgatt tttattatta aataagttat aaaaaaaata agtgtataca |
| 8521 | aattttaaag tgactcttag gttttaaaac gaaaattctt attcttgagt aactctttcc |
| 8581 | tgtaggtcag gttgctttct caggtatagc atgaggtcgc tcgtttaaac aaaaccgctg |
| 8641 | cagcaaccct tgttacatac agtcggatcc atctgactta ctttccttgc gtctccctgc |
| 8701 | gcgatcttgt tggccatttt ccagatcctc tagaattttt caagggtcga gccgtaggag |
| 8761 | gattctctca gaaggcaaaa acgcatcgaa agcgtgcttt gtaagaatat ttggtatggc |
| 8821 | taaagtaagc aaagccatat cccgatcccg atcccgactc ttattccgat cccttctgcc |
| 8881 | acatcctgca tgtttattcg aataccgaat tagctcatct tcgttatttt catcatccct |
| 8941 | ttctgctata gcaaggacaa gtttttttct agcatctcat cgaaaacttt cctctcccta |
| 9001 | attggccaaa gttttcatat tcatcatcag ttagaaagta taatatcaat cccttacctc |
| 9061 | attacaagtt gtatcacact aaaaaaatca tatataagtc tgtgagagtc ttcaattatt |
| 9121 | tagcgtaaca tatgcggccg cggatctgcc ggtctcccta tagtgagtcg tattaatttc |
| 9181 | gataagccag gttaacctgc attaatgaat cggccaacgc gcggggagag gcggtttgcg |
| 9241 | tattgggcgc tcttccgctt cctcgctcac tgactcgctg cgctcggtcg ttcggctgcg |
| 9301 | gcgagcggta tcagctcact caaaggcggt aatacggtta tccacagaat caggggataa |
| 9361 | cgcaggaaag aacatgtgag caaaaggcca gcaaaaggcc aggaaccgta aaaaggccgc |
| 9421 | gttgctggcg tttttccata ggctccgccc ccctgacgag catcacaaaa atcgacgctc |
| 9481 | aagtcagagg tggcgaaacc cgacaggact ataaagatac caggcgtttc cccctggaag |
| 9541 | ctccctcgtg cgctctcctg ttccgaccct gccgcttacc ggatacctgt ccgcctttct |
| 9601 | cccttcggga agcgtggcgc tttctcaatg ctcacgctgt aggtatctca gttcggtgta |
| 9661 | ggtcgttcgc tccaagctgg gctgtgtgca cgaacccccc gttcagcccg accgctgcgc |
| 9721 | cttatccggt aactatcgtc ttgagtccaa cccggtaaga cacgacttat cgccactggc |
| 9781 | agcagccact ggtaacagga ttagcagagc gaggtatgta ggcggtgcta cagagttctt |
| 9841 | gaagtggtgg cctaactacg gctacactag aaggacagta tttggtatct gcgctctgct |
| 9901 | gaagccagtt accttcggaa aaagagttgg tagctcttga tccggcaaac aaaccaccgc |
| 9961 | tggtagcggt ggtttttttg tttgcaagca gcagattacg cgcagaaaaa aaggatctca |
| 10021 | agaagatcct ttgatctttt cta |

SEQ ID NO.: 9. Starch binding domain (SBD) of A. niger glucoamylase

| | |
|---|---|
| 1 | atggttttat ptgsgsvtst skttataskt ststsstsct tptavavtfd ltatttygcn |
| 61 | iylvgsisql gdwctsdgia lsadkytssd plwyvtvtlp agcsfcykfi ricsddsvcw |
| 121 | csdpnrcytv pqacgtstat vtdtwr |

SEQ ID NO.: 10. MAL23-8c constitutive, glucose-insensitive MAL activator

| | |
|---|---|
| 1 | mgiakqscdc crvrrvkcdr nkpcnrctqr nlnctylqpl kkrgpksira gslkkiaevq |
| 61 | mvsmnnnimt apvvckkvpk nlidqclrly hdnlyviwpm lsyddlhkll eenyedcsty |
| 121 | wflvslsaat lsdlqieiey eegvtftgeq lctlcmlsrq ffddlsnsdi frimtyyclh |
| 181 | rcyaqfadtr tsyrlsceai glikiagfhr eetyeflpfg eqqlrrkvyy lllmterfya |
| 241 | vyikcvtsld ttiapplpev vtdprlsles flevirvftv pgkcfydala tncvddscte |
| 301 | dslkriwnel httsldiepw sygyvdisfs rhwiralawk lvfqmngtkf fsnannahil |
| 361 | veiakdmldd ifltpnnlyd vhgpgipmks levanalvdi vnkydhnmkl eawnilcdvs |
| 421 | kfvfslkhcn hkmfqrfstk cqsalidlpi srplrlndds kdeddiip |

The invention claimed is:

1. A recombinant yeast strain comprising:
a strain of S. cerevisiae capable of co-fermenting maltose and glucose;
an exogenous beta-amylase gene;
wherein the strain of S. cerevisiae expresses an exogenous beta-amylase gene.

2. The strain of claim 1 wherein the beta-amylase gene is in the genome of the strain of S. cerevisiae within at least one of (a) intergenic space between YDR439W and YDR440W or (b) the Dubious Open Reading Frame YMR082c or (c) the Dubious Open Reading Frame YCR022C.

3. The strain of claim 1 wherein the beta-amylase gene has at least 80 percent identity to SEQ. ID NO.: 1.

4. The strain of claim 1 wherein the beta-amylase gene is fused to a DNA sequence encoding a polypeptide comprising an amino acid sequence at least 85 percent identical to SEQ ID. NO.: 9.

5. The strain of claim 1 further comprising an exogenous glucoamylase gene from P. oxalicum.

6. The strain of claim 5 wherein the glucoamylase gene from P. oxalicum is in the genome of the strain of S. cerevisiae within one of (a) intergenic space between YDR439W and YDR440W or (b) the Dubious Open Reading Frame YMR082c.

7. The strain of claim 5 wherein the glucoamylase gene from P. oxalicum comprises a sequence having at least 80 percent identity to SEQ. ID NO.: 3.

8. The strain of claim 1 further comprising an exogenous maltogenic alpha-amylase gene from L. plantarum S21.

9. The strain of claim 8 wherein the exogenous maltogenic alpha-amylase gene from L. plantarum S21 is in the genome of the strain of S. cerevisiae within the Dubious Open Reading Frame YCR022C.

10. The strain of claim 8 wherein the maltogenic alpha-amylase gene from L. plantarum S21 comprises a sequence having at least 80% identity to SEQ. ID NO.: 5.

11. The strain of claim 1 for use in a medium comprising starch with a DP4+ component with a weight of approximately 21.5 percent of total volume in corn mash with approximately 33.53 percent weight-to-weight solids without added exogenous glucoamylase; wherein the DP4+ component comprises a weight of no more than 10.7 percent of total volume after 12 hours of fermentation.

12. The strain of claim 5 for use in a medium comprising starch with a DP4+ component with a weight of approximately 21.1 percent of total volume in corn mash with approximately 33.62 percent weight-to-weight solids without added exogenous glucoamylase.

13. The strain of claim 12 wherein the DP4+ component comprises a weight of no more than approximately 9.4 percent of total volume after 12 hours of fermentation.

14. The strain of claim 12 wherein the medium comprises ethanol with a weight of no less than approximately 13.4 percent of total volume after 24 hours of fermentation.

15. The strain of claim 5 for use in a medium comprising starch with a DP4+ component with a weight of approximately 21.1 percent of total volume in corn mash with approximately 33.62 percent weight-to-weight solids with exogenous glucoamylase at a ratio of 0.015 percent weight-to-weight solids.

16. The strain of claim 15 wherein the medium comprises ethanol with a weight of no less than approximately 11.1 percent of total volume after 24 hours of fermentation.

17. The strain of claim 15 wherein the medium comprises glucose with a weight of no more than approximately 0.3 percent of total volume after 24 hours of fermentation.

18. The strain of claim 1 wherein co-fermenting maltose and glucose comprises simultaneous fermentation of maltose and glucose.

19. The strain of claim 1 wherein co-fermenting maltose and glucose comprises accelerated transition from fermentation of glucose to fermentation of maltose.

20. A vector comprising an exogenous beta-amylase gene with a sequence having at least 80 percent homology and/or identity to SEQ. ID NO.: 1.


SEQUENCE LISTING (16 sequences)

SEQ ID NO: 1 - DNA, 1255 bp, synthetic construct
  source 1..1255: mol_type = other DNA
agccgtcaacggccaatcatttaactcaaactataaaacttaccttatggccccactaaaaaaagtgactgagttcactacatgggaagcgttcgaaaacgatttgagaaaagcgaagcaaaatggtttttacgcagtaacggttgacttctggtggggtgacatggagaaaaacggggatcagcagttcgatttctcatacgcgcagaggtttgcccaagctgcgagaaacgcaggcattaaaatggtacctattataagcacgcatcaatgcggaggtaacgtcggcgacgactgcaatacgccgctacctagctggatttggaacaccaagacggatgatagcctatattttaaatccgaaacgggcactgtgaacaaagaaaccgttaacccgctagccacagatgtgataaccaagcaatacggtgagctatacactgcctttgcgcaggcattagcaccctataaagacgtcattccgaaggtctacctgagcggagggccagcaggcgagctaaggtatcctagctatacagctgccgacggaaccggctatccctctcgtggaaaatttcaagcatacaccgactttgccaagtccaaattccagatgtgggcggtaaataaatatggttctttagccggagtcaaccaggcgtggggactgagtttgaccagtactagccagatcctacctccgagtgatggtaaccaattcttaaaggatggctataatactaactacggcaaagacttcctggaatggtatcagggcgtattacaggaccatgcaaagaggataggcgctttagcccatcaagcatttgatcctgtcttcaatgtgcctgtcggagctaagatagcaggaattcattggcagtacaacaaccccacaatgccccacgctgcggaaaagcccgccggatacaataactacagtacactattagactcttttaagactgcgaaactggatttaacctttacctgtctagaaatggttgacagtggcacgtatcccgaatattccatgccaaaaaccctagttaaagaagtcgctagtcttgcgaacgcgaaaggaattgttcttaatggagagaacgcacttagtataggcagtgaggagcagtacaagcgtgccgcggaaatgacttttaattacaatttcgcggggttcaccttgctaagattttacgacgttataaataactccacgaggatgtcccaattcaatcaacacctaaacatcaag

SEQ ID NO: 2 - protein, 418 aa, synthetic construct
AVNGQSFNSNYKTYLMAPLKKVTEFTTWEAFENDLRKAKQNGFYAVTVDFWWGDMEKNGDQQFDFSYAQRFAQAARNAGIKMVPIISTHQCGGNVGDDCNTPLPSWIWNTKTDDSLYFKSETGTVNKETVNPLATDVITKQYGELYTAFAQALAPYKDVIPKVYLSGGPAGELRYPSYTAADGTGYPSRGKFQAYTDFAKSKFQMWAVNKYGSLAGVNQAWGLSLTSTSQILPPSDGNQFLKDGYNTNYGKDFLEWYQGVLQDHAKRIGALAHQAFDPVFNVPVGAKIAGIHWQYNNPTMPHAAEKPAGYNNYSTLLDSFKTAKLDLTFTCLEMVDSGTYPEYSMPKTLVKEVASLANAKGIVLNGENALSIGSEEQYKRAAEMTFNYNFAGFTLLRFYDVINNSTRMSQFNQHLNIK

SEQ ID NO: 3 - DNA, 1851 bp, synthetic construct
  source 1..1851: mol_type = other DNA
gccccacaattgtcccccagggctacttctctagattcctggttatccagcgaaactactttttctttgaacggtattctcgccaacatcggttcttctggtgcttactctaagtctgctgcctctggtgccgtcatcgcttccccttctactagcaaccccgattactattatacctggaccagagacgcagcgttaactttgaaagccttagttgatattttccgtaatggcaatttgggtctacaaaccgttatcgaacaatatgttaatgcacaggctaaattgcaaactgtctctaatccttccggaggtttgtccgacggtgcaggtttgggagaacctaagttcaatgttgacttgtctgctttcactggtgcttggggtagaccacaaagagatggcccggctctacgggctatagcactaatcgatttcggcaattggctgatagataacggatataaatcttacgcggtgaacaacgtttggccaatcgtaaggaacgatttggcctatgttgcccagtactggtcacagtccggcttcgacctatgggaagaagtgaattctatgtctttctttacagttgctaaccaacatcgttcattagtcgaaggatcagctttcgcatctcgtgtcggtgccagctgttctggttgtgactctcaagctcctcagattttgtgttacatgcaatctttttggactgggagttatattaatgccaatacgggtggtggtagatccggtaaagattctaacactattttagcctcgatacatacttttgatcctgctgcttcttgtgatgacgttaccttccaaccatgctcaagtagagctttggctaaccacaaggtctataccgattctttcagatccgtttacgcgttaaactccggtatagcccaaggtaaggccgtttctgtaggtcgttacccagaagatagttactacggtggcaacccatggtttttatcaaacttagcagctgctgagcaactttatgatgctatctaccaatggaacaagattggttccatcactatcacctcgacctcgcttgcatttttcaaggatgtttatccgtctgccgctaccggtacctatgcttctgggtccacaacctttaatgctattatttctgcagtaaagacatatgctgacggctatgtcagtattgttcaatcccactcctatgcgaatggttcgttgtcagaacaattcgacagaaccactggtttgtccatcagtgctcgcgatttaacatggtcttatgcggcgctgttgactgcaaatgacagaagaaatggcgttgtccctccatcgtggggcgcaagttccgctaattcgatacctggttcatgcagcatgggttctgccacaggttcctacgctactccatctgttggttcatggccagcaacacttacttcaggtacagctgcaccttccagtacatcaactactaccaaggctccaactaccaccacggccaccacaacaacttccgccggttcctgtactacaccaaccgcagtggctgttactttcgatgaaattgctacgacgacatttggtgaaaacgtctacttggtaggaagcattagccaattaggtaactggaatacagccaacggtatcccactgtctgcttcaaagtacacctcttcaaatccattatggtacgccactgtgaacttgcccgctggcactacttttcaatacaaatattttagaaaggaatctgatggttccatcaaatgggagtcagacccaaacagatcttacactgttccagccaaatgtggtactactacagccacagaaaatgatacttggagataa

SEQ ID NO: 4 - protein, 616 aa, synthetic construct
APQLSPRATSLDSWLSSETTFSLNGILANIGSSGAYSKSAASGAVIASPSTSNPDYYYTWTRDAALTLKALVDIFRNGNLGLQTVIEQYVNAQAKLQTVSNPSGGLSDGAGLGEPKFNVDLSAFTGAWGRPQRDGPALRAIALIDFGNWLIDNGYKSYAVNNVWPIVRNDLAYVAQYWSQSGFDLWEEVNSMSFFTVANQHRSLVEGSAFASRVGASCSGCDSQAPQILCYMQSFWTGSYINANTGGGRSGKDSNTILASIHTFDPAASCDDVTFQPCSSRALANHKVYTDSFRSVYALNSGIAQGKAVSVGRYPEDSYYGGNPWFLSNLAAAEQLYDAIYQWNKIGSITITSTSLAFFKDVYPSAATGTYASGSTTFNAIISAVKTYADGYVSIVQSHSYANGSLSEQFDRTTGLSISARDLTWSYAALLTANDRRNGVVPPSWGASSANSIPGSCSMGSATGSYATPSVGSWPATLTSGTAAPSSTSTTTKAPTTTTATTTTSAGSCTTPTAVAVTFDEIATTTFGENVYLVGSISQLGNWNTANGIPLSASKYTSSNPLWYATVNLPAGTTFQYKYFRKESDGSIKWESDPNRSYTVPAKCGTTTATENDTWR

SEQ ID NO: 5 - DNA, 2625 bp, synthetic construct
  source 1..2625: mol_type = other DNA
gattcatacaccacctcaacagacgattcgtctaatgacactgccgacagtgtctctgatggtgtgattttacacgcttggtgttggtctttcaacacaatcaagaacaatttgaagcaaattcacgatgcaggttacactgccgttcaaacctcccctgtcaatgaagtcaaagttggtaattctgctagtaagtctttgaacaactggtactggttataccaaccaacaaagtactcgattggtaactattacttaggtaccgaagctgaattcaagtccatgtgtgcagctgccaaggagtacaacatcagaattattgttgatgctaccttgaatgacaccacaagtgactactcagctatttcggatgaaatcaaatccattagtaattggactcatggcaatacacagatatccaactggtcagacagggaggatgtcacccaaaactctctccttggtttgtatgattggaacactcaaaattcccaagtccaaacatacctaaagaactacttggaacgtctaatatcagatggggcaagcggttttcgttacgatgcagccaaacatatcgaattgccatcacaatacgacggttcatatggttccaatttttggccaaatatcactgacaatggtagtgaattccaatatggcgaagttttgcaagattctatttccaaagaatccgattacgctaattacatgtcagtaacagcctctaattatggtaatactattagaaatgccctgaaaaacagagatttcactgctagcacattacaaaatttcaatatttctgtccccgctagcaagttggttacttgggttgaatctcatgacaactatgcaaacgatgaccaagtttctacctggatgaatagttccgatattaaactaggttgggccgtagtggcctcaagatctggaagtgttccattatttttcgacagaccagttgacggtggtaatggtacccgttttcctggatctagcgaaattggtgacgccggttcttcgctttattatgacaaggctgttgtggcggttaacaagttccacaacgccatggctggtcaatctgaatacatttcaaacccaaacggtaacaccaaaatttttgaaaacgaaagaggttctaagggtgtcgttttcgctaatgcttcggatggcagctattctctatctgttaagacatctcttgctgacggtacctacgaaaataaggccggaagtgacgagttcactgttaaaaacggttatttgacaggtactatccaaggtagagaagtagtcgtattatatggcgatccaacttcaagctcgtcctcgtctaccactactgaaactaagaaggtgtattttgaaaaaccatcctcctggggttccacagtctatgcctatgtctacaacaaaaacactaataaggctataaccagcgcatggccaggtaaagagatgactgctttaggtaatgatgagtataaattagacctggatacagatgaagatgattccgacttggcagtaattttcaccgatgggaccaaccaaactcctgcagccaacaaggctgggttcaccttcacagcagacgcgacgtacgatcagaacggtgttgttaagacctctgactcatcttcgtcgtcctccactaccaccgaaacaaaaaaagtgtattttgaaaagccttcatcttgggggtccactgtctacgcctacgtttataataaaaacacgaacaaagctatcaccagtgcttggcccggtaaggaaatgaccgctcttggaaatgacgaatataaattggatttggatactgatgaagatgatagtgatctagctgttatctttactgatggtacaaaccaaacgccggcagctaacaaggcaggtttcacttttaccgctgatgccacttatgatcaaaacggtgtggttaagacatctgacagttcttcatcatcttccagtacaactacggaaactaagaaagtttacttcgaaaagccatcttcgtggggctctacggtttacgcttatgtttataacaagaatacaaataaagcaattacttccgcttggcctggtaaggaaatgactgcgttaggcaacgacgaatacaagttagatttagataccgatgaagatgatagtgatttggctgtgatcttcactgatggaaccaaccagactccagctgctaacaaagcaggctttacctttactgctgatgccacttatgaccagaatggtgttgtcaagacctccgatagctcctcttcctcgtcaactactacagaaacgaagaaggtttactttgagaagccaagtagttggggttctacagtttatgcttacgtatacaataaaaatactaataaagcgatcactagcgcctggccaggtaaagaaatgacagctttgggcaatgacgaatacaaattggaccttgacactgacgaggacgactccgatttggctgttatatttaccgatggtactaatcaaacgcctgctgcaaataaagctggtttcacatttaccgccgatgctacttacgatcagaacggtgtcgtcaaaacatctgattcttcgtccacctcttctacatcataa

SEQ ID NO: 6 - protein, 874 aa, synthetic construct
DSYTTSTDDSSNDTADSVSDGVILHAWCWSFNTIKNNLKQIHDAGYTAVQTSPVNEVKVGNSASKSLNNWYWLYQPTKYSIGNYYLGTEAEFKSMCAAAKEYNIRIIVDATLNDTTSDYSAISDEIKSISNWTHGNTQISNWSDREDVTQNSLLGLYDWNTQNSQVQTYLKNYLERLISDGASGFRYDAAKHIELPSQYDGSYGSNFWPNITDNGSEFQYGEVLQDSISKESDYANYMSVTASNYGNTIRNALKNRDFTASTLQNFNISVPASKLVTWVESHDNYANDDQVSTWMNSSDIKLGWAVVASRSGSVPLFFDRPVDGGNGTRFPGSSEIGDAGSSLYYDKAVVAVNKFHNAMAGQSEYISNPNGNTKIFENERGSKGVVFANASDGSYSLSVKTSLADGTYENKAGSDEFTVKNGYLTGTIQGREVVVLYGDPTSSSSSSTTTETKKVYFEKPSSWGSTVYAYVYNKNTNKAITSAWPGKEMTALGNDEYKLDLDTDEDDSDLAVIFTDGTNQTPAANKAGFTFTADATYDQNGVVKTSDSSSSSSTTTETKKVYFEKPSSWGSTVYAYVYNKNTNKAITSAWPGKEMTALGNDEYKLDLDTDEDDSDLAVIFTDGTNQTPAANKAGFTFTADATYDQNGVVKTSDSSSSSSSTTTETKKVYFEKPSSWGSTVYAYVYNKNTNKAITSAWPGKEMTALGNDEYKLDLDTDEDDSDLAVIFTDGTNQTPAANKAGFTFTADATYDQNGVVKTSDSSSSSSTTTETKKVYFEKPSSWGSTVYAYVYNKNTNKAITSAWPGKEMTALGNDEYKLDLDTDEDDSDLAVIFTDGTNQTPAANKAGFTFTADATYDQNGVVKTSDSSSTSSTS

SEQ ID NO: 7 - DNA, 9456 bp, synthetic construct
  source 1..9456: mol_type = other DNA
  misc_feature complement(1140..1147): note = NotI
  CDS 2586..2861: note = Ost1-Pro-a factor signal peptide; protein_id = 11; translation = MRQVWFSWIVGLFLCFFNVSSAAPVNTTTEDETAQIPAEAVIGY SDLEGDFDVAVLPFSNSTNNGLLFINTTIASIAAKEEGVSLDKREAEA
  misc_feature 1151..1650: note = UPS_NLS9
  misc_feature 1651..1658: note = PmeI
  misc_feature 1659..1848: note = Terminator CYC1
  misc_feature 1849..1858: note = SwaI site Del
  misc_feature 1859..2585: note = Promoter HOR7
  CDS 2862..4712: note = PoGA15A; protein_id = 12; translation = APQLSPRATSLDSWLSSETTFSLNGILANIGSSGAYSKSAASGA VIASPSTSNPDYYYTWTRDAALTLKALVDIFRNGNLGLQTVIEQYVNAQAKLQTVSNP SGGLSDGAGLGEPKFNVDLSAFTGAWGRPQRDGPALRAIALIDFGNWLIDNGYKSYAV NNVWPIVRNDLAYVAQYWSQSGFDLWEEVNSMSFFTVANQHRSLVEGSAFASRVGASC SGCDSQAPQILCYMQSFWTGSYINANTGGGRSGKDSNTILASIHTFDPAASCDDVTFQ PCSSRALANHKVYTDSFRSVYALNSGIAQGKAVSVGRYPEDSYYGGNPWFLSNLAAAE QLYDAIYQWNKIGSITITSTSLAFFKDVYPSAATGTYASGSTTFNAIISAVKTYADGY VSIVQSHSYANGSLSEQFDRTTGLSISARDLTWSYAALLTANDRRNGVVPPSWGASSA NSIPGSCSMGSATGSYATPSVGSWPATLTSGTAAPSSTSTTTKAPTTTTATTTTSAGS CTTPTAVAVTFDEIATTTFGENVYLVGSISQLGNWNTANGIPLSASKYTSSNPLWYAT VNLPAGTTFQYKYFRKESDGSIKWESDPNRSYTVPAKCGTTTATENDTWR
  misc_feature 4713..4718: note = ApaI
  misc_feature 4719..5146: note = Terminator PGK1
  misc_feature 5147..5323: note = CPS1 Terminator
  misc_feature 5324..5764: note = Bamy_SBD-An
  CDS complement(7019..7096): note = GLM-SP; protein_id = 13; translation = MIRLTVFLTAVFAAVASCVPVELDKR
  misc_feature complement(7097..7832): note = Promoter CCW12
  misc_feature 7833..7840: note = SwaI
  misc_feature 7841..8035: note = Terminator ADH1
  misc_feature 8036..8043: note = PmeI
  misc_feature 8044..8543: note = DWS_NLS9
  misc_feature 8547..8554: note = Not1
  misc_feature 8828..9456: note = colE1
  CDS 149..811: note = Amp Resistance gene
  CDS complement(5765..7019): note = Bamy
  misc_feature 1849..1858: note = SwaI site Del
cggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcggccgcatcctgacattttgttgttttccttgcgtgcccaatgttgccagcgtaaaagttatcgtaaacgttatcgtaaacgttatccccgttttgtaaactttattgtcttcgtaaaaaatagaaaccaacacttaaaattggtcccataaaaaattgagtcagttccttcatcgtggtacagtcagagtattctaaattaattaatttactggtaatttaataacatgaagaagaaagattggatattgaagttctccaccaggcacatataacgctctaaataatatttagatgttgcactaaagtccacgatgaagaataaaaagcatcacttgatcgttcaatgagccaatcttggaaattttgacacctctattaaccagaacatccagcttgttgagaactgtaactttgaaactcaaataactcaaggaacgggagtctttcaagattttggaataatttgaccactttgtttttaaacaattcgagtgaaggccatgcgcgtttaaaccttcgagcgtcccaaaaccttctcaagcaaggttttcagtataatgttacatgcgtacacgcgtttgtacagaaaaaaaagaaaaatttgaaatataaataacgttcttaatactaacataactataaaaaaataaatagggacctagacttcaggttgtctaactccttccttttcggttagagcggatatttcgaaatctttcgattagcacgcacacacatcacatagactgcgtcataaaaatacactacggaaaaaccataaagagcaaagcgatacctacttggaaggaaaaggagcacgcttgtaagggggatgggggctaagaagtcattcactttcttttcccttcgcggtccggacccgggacccctcctctccccgcacaatttcttcctttcatatcttccttttattcctatcccgttgaagcaaccgcactatgactaaatggtgctggacatctccatggctgtgacttgtgtgtatctcacagtggtaacggcaccgtggctcggaaacggttccttcgtgacaattctagaacaggggctacagtctcgataatagaataataagcgcatttttgttagcgccgccgcggcgcccgtttcccaatagggaggcgcagtttatcggcggagctttacttcttcctatttgggtaagcccctttctgttttcggccagtggttgctgcaggctgcgccggagaacatagtgataagggatgtaactttcgatgagagaattagcaagcggaaaaaaaactatggctagctgggagttgtttttcaatcatataaaagggagaaattgttgctcactatgtgacagtttctgggacgtcttaacttttattgcagaggactatcaaatcatacagatattgtcaaaaaaaaaaaaaaagactaataataaaaaatgagacaagtctggttctcttggattgttggtttattcttatgtttttttaacgtttcgtcagctgctccagttaacactacaactgaagacgagaccgctcaaattccagctgaggcagtcattggttacagtgatttggaaggtgactttgacgtggcagttttaccatttagtaactctaccaataatgggttgctattcattaatactaccatcgcctcaattgctgctaaggaggaaggtgttagcttggataaaagggaagccgaagctgccccacaattgtcccccagggctacttctctagattcctggttatccagcgaaactactttttctttgaacggtattctcgccaacatcggttcttctggtgcttactctaagtctgctgcctctggtgccgtcatcgcttccccttctactagcaaccccgattactattatacctggaccagagacgcagcgttaactttgaaagccttagttgatattttccgtaatggcaatttgggtctacaaaccgttatcgaacaatatgttaatgcacaggctaaattgcaaactgtctctaatccttccggaggtttgtccgacggtgcaggtttgggagaacctaagttcaatgttgacttgtctgctttcactggtgcttggggtagaccacaaagagatggcccggctctacgggctatagcactaatcgatttcggcaattggctgatagataacggatataaatcttacgcggtgaacaacgtttggccaatcgtaaggaacgatttggcctatgttgcccagtactggtcacagtccggcttcgacctatgggaagaagtgaattctatgtctttctttacagttgctaaccaacatcgttcattagtcgaaggatcagctttcgcatctcgtgtcggtgccagctgttctggttgtgactctcaagctcctcagattttgtgttacatgcaatctttttggactgggagttatattaatgccaatacgggtggtggtagatccggtaaagattctaacactattttagcctcgatacatacttttgatcctgctgcttcttgtgatgacgttaccttccaaccatgctcaagtagagctttggctaaccacaaggtctataccgattctttcagatccgtttacgcgttaaactccggtatagcccaaggtaaggccgtttctgtaggtcgttacccagaagatagttactacggtggcaacccatggtttttatcaaacttagcagctgctgagcaactttatgatgctatctaccaatggaacaagattggttccatcactatcacctcgacctcgcttgcatttttcaaggatgtttatccgtctgccgctaccggtacctatgcttctgggtccacaacctttaatgctattatttctgcagtaaagacatatgctgacggctatgtcagtattgttcaatcccactcctatgcgaatggttcgttgtcagaacaattcgacagaaccactggtttgtccatcagtgctcgcgatttaacatggtcttatgcggcgctgttgactgcaaatgacagaagaaatggcgttgtccctccatcgtggggcgcaagttccgctaattcgatacctggttcatgcagcatgggttctgccacaggttcctacgctactccatctgttggttcatggccagcaacacttacttcaggtacagctgcaccttccagtacatcaactactaccaaggctccaactaccaccacggccaccacaacaacttccgccggttcctgtactacaccaaccgcagtggctgttactttcgatgaaattgctacgacgacatttggtgaaaacgtctacttggtaggaagcattagccaattaggtaactggaatacagccaacggtatcccactgtctgcttcaaagtacacctcttcaaatccattatggtacgccactgtgaacttgcccgctggcactacttttcaatacaaatattttagaaaggaatctgatggttccatcaaatgggagtcagacccaaacagatcttacactgttccagccaaatgtggtactactacagccacagaaaatgatacttggagataagggcccattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatccagtttttttttcccattcgatatttctatgttcgggttcagcgtattttaagtttaataactcgaaaattctgcgttcgttaaagctttcgagaaggatattatttcgaaataaaccgtgttgtgtaagcttgaagcctttttgcgctgccaatattcttatccatctattgtactctttagatccagtatagtgtattcttcctgctccaagttcatcccacttgcaacaaaaatttgacacttgatttgacacttctttttttttttatttatgttttgtttaactgacggtgttttttttttttacgttatactgatacatatatagacgtgatttatttaaataaaaagttctatgaaaaaaaaaaaaaattaaaaaaaaaaatctttgactattcaatcattgcgcttacctccaagtgtctgtaaccgtggccgtggaggtcccgcatgcctgcggaaccgtatattctctgtttgggtcagattcccactcaacagagtcgtcagattcgatcctaataaatttatattcgaaggactcacctgcgggtagtgtgacggtgacataccaaagagggtcagaactggtgtacttatctgcagacagagcaatcccatcagaggtttcccaatcccccaactgagagatactcccgacaagatagatgttttctccgtaggtagtggtggccgttaggtcgaatgtgacagcaacagcggtcggagtagtgcatgaagtgcttgatgtggaagttgatgttttgctggctgttgcagtcgtcttagacgttgacgttacgctacctgatcctgtcggagtcgcggttgttgtcgtgccgccggtggccttgatgtttaggtgttgattgaattgggacatcctcgtggagttatttataacgtcgtaaaatcttagcaaggtgaaccccgcgaaattgtaattaaaagtcatttccgcggcacgcttgtactgctcctcactgcctatactaagtgcgttctctccattaagaacaattcctttcgcgttcgcaagactagcgacttctttaactagggtttttggcatggaatattcgggatacgtgccactgtcaaccatttctagacaggtaaaggttaaatccagtttcgcagtcttaaaagagtctaatagtgtactgtagttattgtatccggcgggcttttccgcagcgtggggcattgtggggttgttgtactgccaatgaattcctgctatcttagctccgacaggcacattgaagacaggatcaaatgcttgatgggctaaagcgcctatcctctttgcatggtcctgtaatacgccctgataccattccaggaagtctttgccgtagttagtattatagccatcctttaagaattggttaccatcactcggaggtaggatctggctagtactggtcaaactcagtccccacgcctggttgactccggctaaagaaccatatttatttaccgcccacatctggaatttggacttggcaaagtcggtgtatgcttgaaattttccacgagagggatagccggttccgtcggcagctgtatagctaggataccttagctcgcctgctggccctccgctcaggtagaccttcggaatgacgtctttatagggtgctaatgcctgcgcaaaggcagtgtatagctcaccgtattgcttggttatcacatctgtggctagcgggttaacggtttctttgttcacagtgcccgtttcggatttaaaatataggctatcatccgtcttggtgttccaaatccagctaggtagcggcgtattgcagtcgtcgccgacgttacctccgcattgatgcgtgcttataataggtaccattttaatgcctgcgtttctcgcagcttgggcaaacctctgcgcgtatgagaaatcgaactgctgatccccgtttttctccatgtcaccccaccagaagtcaaccgttactgcgtaaaaaccattttgcttcgcttttctcaaatcgttttcgaacgcttcccatgtagtgaactcagtcactttttttagtggggccataaggtaagttttatagtttgagttaaatgattggccgttgacggctctcttgtcaagctcaacgggaacacaagatgcgacagctgcgaaaacagcggttaagaagacagtcaatctgatcattattgatatagtgtttaagcgaatgacagaagattaatttcttggtatgttaggaaagaataaaggagaataagaataattagaacaatgtaggatggaaagaaagattatcaagcatgccgactttatatacttgaacggaggcaaaggatgcaaaattttctcacatttctttctgccgttatgttggaagtaagactcccattatcgcaatactgcaacacgaatatgcaaaatttgctgagttatcgcagatagttgttgcaaagatagcggcgtaggtggccgcgaaatggggaattccaaaacaaacggtttttttactcctgagaaatacttgtacgggataatccagggcctaccacccacgcttcgaggattggcttttatttttttttttttggtggcgttttatttctttcccgctttctgggacttgtgcggagttttgagaggggcgcgcggcaaaggattcccaaaacggaaatcagacgccaatagccagcactcaaagcagttctggacccattgcgattttcccatttggttcttgcgcgtgctgattccgacacgcgcgtctataaatagcatgaagtatccgcacaccgcagcgttagtgaggtgagggtggcagcaagctaattcccgcatctggaatctgaactgccccttttggactaaccgtgtggttcatgggtgggcgaagtgcgcaacctcgaaggttttcttttgcgtgatttaaatgtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcgtttaaacttaaaaacatttctttctttatccaattgaattatgccattcgatattaaatacgacaacaggaacactgaagaacgggatactccccccttgtgaatgtgctacaataaacagaatatcggcacccggttatttcttctctccaattcttactaaattagcgataaattctaccctttccataacgggccgttcttctgcaagagaatttttcctacttgaataggcatatttgaatctgaaaagtagtcacagatagtgaactctgctttattaataaaattccaggatgtactcttggagatcgaatcaagttcctagaaatcctttgagaaagaagcggtgaatacccgtaggtaggattttttcacgtaattaaaccgccttatcttatttgttgccagaaaactagaggatggaataaacatgttccctaatggtgtggcatttacctgtctgtctagtttctgttagtgcattccaatgtcagctgcttcgcttatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttcta

SEQ ID NO: 8 - DNA, 10043 bp, synthetic construct
  source 1..10043: mol_type = other DNA
  CDS 149..811: note = Amp Resistance gene
  misc_feature complement(1140..1147): note = NotI
  misc_feature 1151..1650: note = UPS_NLS7
  misc_feature 1651..1658: note = PmeI
  misc_feature 1659..1850: note = Terminator CYC1
  misc_feature 1851..1860: note = SwaI site del
  misc_feature 1861..2587: note = Promoter HOR7
  CDS 2588..2665: note = GLM-SP; protein_id = 14; translation = MIRLTVFLTAVFAAVASCVPVELDKR
  CDS 2666..5290: note = MALPS21; protein_id = 15; translation = DSYTTSTDDSSNDTADSVSDGVILHAWCWSFNTIKNNLKQIHDA GYTAVQTSPVNEVKVGNSASKSLNNWYWLYQPTKYSIGNYYLGTEAEFKSMCAAAKEY NIRIIVDATLNDTTSDYSAISDEIKSISNWTHGNTQISNWSDREDVTQNSLLGLYDWN TQNSQVQTYLKNYLERLISDGASGFRYDAAKHIELPSQYDGSYGSNFWPNITDNGSEF QYGEVLQDSISKESDYANYMSVTASNYGNTIRNALKNRDFTASTLQNFNISVPASKLV TWVESHDNYANDDQVSTWMNSSDIKLGWAVVASRSGSVPLFFDRPVDGGNGTRFPGSS EIGDAGSSLYYDKAVVAVNKFHNAMAGQSEYISNPNGNTKIFENERGSKGVVFANASD GSYSLSVKTSLADGTYENKAGSDEFTVKNGYLTGTIQGREVVVLYGDPTSSSSSSTTT ETKKVYFEKPSSWGSTVYAYVYNKNTNKAITSAWPGKEMTALGNDEYKLDLDTDEDDS DLAVIFTDGTNQTPAANKAGFTFTADATYDQNGVVKTSDSSSSSSTTTETKKVYFEKP SSWGSTVYAYVYNKNTNKAITSAWPGKEMTALGNDEYKLDLDTDEDDSDLAVIFTDGT NQTPAANKAGFTFTADATYDQNGVVKTSDSSSSSSSTTTETKKVYFEKPSSWGSTVYA YVYNKNTNKAITSAWPGKEMTALGNDEYKLDLDTDEDDSDLAVIFTDGTNQTPAANKA GFTFTADATYDQNGVVKTSDSSSSSSTTTETKKVYFEKPSSWGSTVYAYVYNKNTNKA ITSAWPGKEMTALGNDEYKLDLDTDEDDSDLAVIFTDGTNQTPAANKAGFTFTADATY DQNGVVKTSDSSSTSSTS
  misc_feature 5291..5718: note = Terminator PGK1
  misc_feature 5719..5724: note = ApaI
  misc_feature 5725..5901: note = CPS1 Terminator
  misc_feature 5902..6342: note = Bamy_SBD-An
  CDS complement(6343..7597): note = Bamy
  CDS complement(7597..7674): note = GLM-SP; protein_id = 16; translation = MIRLTVFLTAVFAAVASCVPVELDKR
  misc_feature complement(7675..8410): note = Promoter CCW12
  misc_feature 8411..8416: note = ApaI
  misc_feature 8420..8427: note = SwaI
  misc_feature 8428..8622: note = Terminator ADH1
  misc_feature 8623..8630: note = PmeI
  misc_feature 8631..9130: note = DWS_NLS7
  misc_feature 9134..9141: note = Not1
  misc_feature 9415..10043: note = colE1
cggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcggccgcatcccattttgagcgagagaacccatttttctatacaaatttcactagagcacggccgttacatttagtaatagccaataagggttttttatcgattagtgttccctgcgctccttaacatcatacaaccgagtccttgacatggaaatagtaggcaagtaaaccaaagtcctttcttcaaaagtagaaaacttgagcacttatttcctgcgcatgtcatatgttaattttccttaactgcgctgaatacgtcctgtcaattcaaatatatcacgttttgagcagccctaaagaagaaaacctcaacagcagtattactattacaatcaaacaactttagtgccgcgtgataccgggggttgaagtgggtgcattgagccgtattcttcttccccgtaagaaagttatgtatcctttttactgcgttgtaatagcttctgaaaacctaaaaaatgaacgctatgtagctcatatccgtttcgcataagtaagaataactacttgtttaaaccttcgagcgtcccaaaaccttctcaagcaaggttttcagtataatgttacatgcgtacacgcgtttgtacagaaaaaaaagaaaaatttgaaatataaataacgttcttaatactaacataactattaaaaaaaataaatagggacctagacttcaggttgtctaactccttccttttcggttagagcggatatttcgaaatctttcgattagcacgcacacacatcacatagactgcgtcataaaaatacactacggaaaaaccataaagagcaaagcgatacctacttggaaggaaaaggagcacgcttgtaagggggatgggggctaagaagtcattcactttcttttcccttcgcggtccggacccgggacccctcctctccccgcacaatttcttcctttcatatcttccttttattcctatcccgttgaagcaaccgcactatgactaaatggtgctggacatctccatggctgtgacttgtgtgtatctcacagtggtaacggcaccgtggctcggaaacggttccttcgtgacaattctagaacaggggctacagtctcgataatagaataataagcgcatttttgttagcgccgccgcggcgcccgtttcccaatagggaggcgcagtttatcggcggagctttacttcttcctatttgggtaagcccctttctgttttcggccagtggttgctgcaggctgcgccggagaacatagtgataagggatgtaactttcgatgagagaattagcaagcggaaaaaaaactatggctagctgggagttgtttttcaatcatataaaagggagaaattgttgctcactatgtgacagtttctgggacgtcttaacttttattgcagaggactatcaaatcatacagatattgtcaaaaaaaaaaaaaaagactaataataaaaaatgatcagattgactgtcttcttaaccgctgttttcgcagctgtcgcatcttgtgttcccgttgagcttgacaagagagattcatacaccacctcaacagacgattcgtctaatgacactgccgacagtgtctctgatggtgtgattttacacgcttggtgttggtctttcaacacaatcaagaacaatttgaagcaaattcacgatgcaggttacactgccgttcaaacctcccctgtcaatgaagtcaaagttggtaattctgctagtaagtctttgaacaactggtactggttataccaaccaacaaagtactcgattggtaactattacttaggtaccgaagctgaattcaagtccatgtgtgcagctgccaaggagtacaacatcagaattattgttgatgctaccttgaatgacaccacaagtgactactcagctatttcggatgaaatcaaatccattagtaattggactcatggcaatacacagatatccaactggtcagacagggaggatgtcacccaaaactctctccttggtttgtatgattggaacactcaaaattcccaagtccaaacatacctaaagaactacttggaacgtctaatatcagatggggcaagcggttttcgttacgatgcagccaaacatatcgaattgccatcacaatacgacggttcatatggttccaatttttggccaaatatcactgacaatggtagtgaattccaatatggcgaagttttgcaagattctatttccaaagaatccgattacgctaattacatgtcagtaacagcctctaattatggtaatactattagaaatgccctgaaaaacagagatttcactgctagcacattacaaaatttcaatatttctgtccccgctagcaagttggttacttgggttgaatctcatgacaactatgcaaacgatgaccaagtttctacctggatgaatagttccgatattaaactaggttgggccgtagtggcctcaagatctggaagtgttccattatttttcgacagaccagttgacggtggtaatggtacccgttttcctggatctagcgaaattggtgacgccggttcttcgctttattatgacaaggctgttgtggcggttaacaagttccacaacgccatggctggtcaatctgaatacatttcaaacccaaacggtaacaccaaaatttttgaaaacgaaagaggttctaagggtgtcgttttcgctaatgcttcggatggcagctattctctatctgttaagacatctcttgctgacggtacctacgaaaataaggccggaagtgacgagttcactgttaaaaacggttatttgacaggtactatccaaggtagagaagtagtcgtattatatggcgatccaacttcaagctcgtcctcgtctaccactactgaaactaagaaggtgtattttgaaaaaccatcctcctggggttccacagtctatgcctatgtctacaacaaaaacactaataaggctataaccagcgcatggccaggtaaagagatgactgctttaggtaatgatgagtataaattagacctggatacagatgaagatgattccgacttggcagtaattttcaccgatgggaccaaccaaactcctgcagccaacaaggctgggttcaccttcacagcagacgcgacgtacgatcagaacggtgttgttaagacctctgactcatcttcgtcgtcctccactaccaccgaaacaaaaaaagtgtattttgaaaagccttcatcttgggggtccactgtctacgcctacgtttataataaaaacacgaacaaagctatcaccagtgcttggcccggtaaggaaatgaccgctcttggaaatgacgaatataaattggatttggatactgatgaagatgatagtgatctagctgttatctttactgatggtacaaaccaaacgccggcagctaacaaggcaggtttcacttttaccgctgatgccacttatgatcaaaacggtgtggttaagacatctgacagttcttcatcatcttccagtacaactacggaaactaagaaagtttacttcgaaaagccatcttcgtggggctctacggtttacgcttatgtttataacaagaatacaaataaagcaattacttccgcttggcctggtaaggaaatgactgcgttaggcaacgacgaatacaagttagatttagataccgatgaagatgatagtgatttggctgtgatcttcactgatggaaccaaccagactccagctgctaacaaagcaggctttacctttactgctgatgccacttatgaccagaatggtgttgtcaagacctccgatagctcctcttcctcgtcaactactacagaaacgaagaaggtttactttgagaagccaagtagttggggttctacagtttatgcttacgtatacaataaaaatactaataaagcgatcactagcgcctggccaggtaaagaaatgacagctttgggcaatgacgaatacaaattggaccttgacactgacgaggacgactccgatttggctgttatatttaccgatggtactaatcaaacgcctgctgcaaataaagctggtttcacatttaccgccgatgctacttacgatcagaacggtgtcgtcaaaacatctgattcttcgtccacctcttctacatcataaattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatccagtttttttttcccattcgatatttctatgttcgggttcagcgtattttaagtttaataactcgaaaattctgcgttcgttaaagctttcgagaaggatattatttcgaaataaaccgtgttgtgtaagcttgaagcctttttgcgctgccaatattcttatccatctattgtactctttagatccagtatagtgtattcttcctgctccaagttcatcccacttgcaacaaaagggcccatttgacacttgatttgacacttctttttttttttatttatgttttgtttaactgacggtgttttttttttttacgttatactgatacatatatagacgtgatttatttaaataaaaagttctatgaaaaaaaaaaaaaattaaaaaaaaaaatctttgactattcaatcattgcgcttacctccaagtgtctgtaaccgtggccgtggaggtcccgcatgcctgcggaaccgtatattctctgtttgggtcagattcccactcaacagagtcgtcagattcgatcctaataaatttatattcgaaggactcacctgcgggtagtgtgacggtgacataccaaagagggtcagaactggtgtacttatctgcagacagagcaatcccatcagaggtttcccaatcccccaactgagagatactcccgacaagatagatgttttctccgtaggtagtggtggccgttaggtcgaatgtgacagcaacagcggtcggagtagtgcatgaagtgcttgatgtggaagttgatgttttgctggctgttgcagtcgtcttagacgttgacgttacgctacctgatcctgtcggagtcgcggttgttgtcgtgccgccggtggccttgatgtttaggtgttgattgaattgggacatcctcgtggagttatttataacgtcgtaaaatcttagcaaggtgaaccccgcgaaattgtaattaaaagtcatttccgcggcacgcttgtactgctcctcactgcctatactaagtgcgttctctccattaagaacaattcctttcgcgttcgcaagactagcgacttctttaactagggtttttggcatggaatattcgggatacgtgccactgtcaaccatttctagacaggtaaaggttaaatccagtttcgcagtcttaaaagagtctaatagtgtactgtagttattgtatccggcgggcttttccgcagcgtggggcattgtggggttgttgtactgccaatgaattcctgctatcttagctccgacaggcacattgaagacaggatcaaatgcttgatgggctaaagcgcctatcctctttgcatggtcctgtaatacgccctgataccattccaggaagtctttgccgtagttagtattatagccatcctttaagaattggttaccatcactcggaggtaggatctggctagtactggtcaaactcagtccccacgcctggttgactccggctaaagaaccatatttatttaccgcccacatctggaatttggacttggcaaagtcggtgtatgcttgaaattttccacgagagggatagccggttccgtcggcagctgtatagctaggataccttagctcgcctgctggccctccgctcaggtagaccttcggaatgacgtctttatagggtgctaatgcctgcgcaaaggcagtgtatagctcaccgtattgcttggttatcacatctgtggctagcgggttaacggtttctttgttcacagtgcccgtttcggatttaaaatataggctatcatccgtcttggtgttccaaatccagctaggtagcggcgtattgcagtcgtcgccgacgttacctccgcattgatgcgtgcttataataggtaccattttaatgcctgcgtttctcgcagcttgggcaaacctctgcgcgtatgagaaatcgaactgctgatccccgtttttctccatgtcaccccaccagaagtcaaccgttactgcgtaaaaaccattttgcttcgcttttctcaaatcgttttcgaacgcttcccatgtagtgaactcagtcactttttttagtggggccataaggtaagttttatagtttgagttaaatgattggccgttgacggctctcttgtcaagctcaacgggaacacaagatgcgacagctgcgaaaacagcggttaagaagacagtcaatctgatcattattgatatagtgtttaagcgaatgacagaagattaatttcttggtatgttaggaaagaataaaggagaataagaataattagaacaatgtaggatggaaagaaagattatcaagcatgccgactttatatacttgaacggaggcaaaggatgcaaaattttctcacatttctttctgccgttatgttggaagtaagactcccattatcgcaatactgcaacacgaatatgcaaaatttgctgagttatcgcagatagttgttgcaaagatagcggcgtaggtggccgcgaaatggggaattccaaaacaaacggtttttttactcctgagaaatacttgtacgggataatccagggcctaccacccacgcttcgaggattggcttttatttttttttttttggtggcgttttatttctttcccgctttctgggacttgtgcggagttttgagaggggcgcgcggcaaaggattcccaaaacggaaatcagacgccaatagccagcactcaaagcagttctggacccattgcgattttcccatttggttcttgcgcgtgctgattccgacacgcgcgtctataaatagcatgaagtatccgcacaccgcagcgttagtgaggtgagggtggcagcaagctaattcccgcatctggaatctgaactgccccttttggactaaccgtgtggttcatgggtgggcgaagtgcgcaacctcgaaggttttcttttgcgtggggccctatatttaaatgtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcgtttaaacaaaaccgctgcagcaacccttgttacatacagtcggatccatctgacttactttccttgcgtctccctgcgcgatcttgttggccattttccagatcctctagaatttttcaagggtcgagccgtaggaggattctctcagaaggcaaaaacgcatcgaaagcgtgctttgtaagaatatttggtatggctaaagtaagcaaagccatatcccgatcccgatcccgactcttattccgatcccttctgccacatcctgcatgtttattcgaataccgaattagctcatcttcgttattttcatcatccctttctgctatagcaaggacaagtttttttctagcatctcatcgaaaactttcctctccctaattggccaaagttttcatattcatcatcagttagaaagtataatatcaatcccttacctcattacaagttgtatcacactaaaaaaatcatatataagtctgtgagagtcttcaattatttagcgtaacatatgcggccgcggatctgccggtctccctatagtgagtcgtattaatttcgataagccaggttaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttcta

SEQ ID NO: 9 - protein, 146 aa, Aspergillus niger
ATGGTTTTATPTGSGSVTSTSKTTATASKTSTSTSSTSCTTPTAVAVTFDLTATTTYGENIYLVGSISQLGDWETSDGIALSADKYTSSDPLWYVTVTLPAGESFEYKFIRIESDDSVEWESDPNREYTVPQACGTSTATVTDTWR

SEQ ID NO: 10 - protein, 468 aa, synthetic construct
MGIAKQSCDCCRVRRVKCDRNKPCNRCTQRNLNCTYLQPLKKRGPKSIRAGSLKKIAEVQMVSMNNNIMTAPVVCKKVPKNLIDQCLRLYHDNLYVIWPMLSYDDLHKLLEENYEDCSTYWFLVSLSAATLSDLQIEIEYEEGVTFTGEQLCTLCMLSRQFFDDLSNSDIFRIMTYYCLHRCYAQFADTRTSYRLSCEAIGLIKIAGFHREETYEFLPFGEQQLRRKVYYLLLMTERFYAVYIKCVTSLDTTIAPPLPEVVTDPRLSLESFLEVIRVFTVPGKCFYDALATNCVDDSCTEDSLKRIWNELHTTSLDIEPWSYGYVDISFSRHWIRALAWKLVFQMNGTKFFSNANNAHILVEIAKDMLDDIFLTPNNLYDVHGPGIPMKSLEVANALVDIVNKYDHNMKLEAWNILCDVSKFVFSLKHCNHKMFQRFSTKCQSALIDLPISRPLRLNDDSKDEDDIIP

SEQ ID NO: 11 - protein, 92 aa, synthetic construct
MRQVWFSWIVGLFLCFFNVSSAAPVNTTTEDETAQIPAEAVIGYSDLEGDFDVAVLPFSNSTNNGLLFINTTIASIAAKEEGVSLDKREAEA

SEQ ID NO: 12 - protein, 616 aa, synthetic construct; identical to SEQ ID NO: 4

SEQ ID NO: 13 - protein, 26 aa, synthetic construct
MIRLTVFLTAVFAAVASCVPVELDKR

SEQ ID NO: 14 - protein, 26 aa, synthetic construct
MIRLTVFLTAVFAAVASCVPVELDKR

SEQ ID NO: 15 - protein, 874 aa, synthetic construct; identical to SEQ ID NO: 6

SEQ ID NO: 16 - protein, 26 aa, synthetic construct
MIRLTVFLTAVFAAVASCVPVELDKR